(19) United States
(12) Patent Application Publication
(10) Pub. No.: US 2014/0328815 A1
(43) Pub. Date: Nov. 6, 2014

(54) Lactic Acid Bacteria And Their Use As Dietary Supplementals For Poultry

(71) Applicant: Nutrition Physiology Company, LLC, Guymon, OK (US)

(72) Inventor: Douglas R. Ware, Chapel Hill, NC (US)

(21) Appl. No.: 14/357,698

(22) PCT Filed: Nov. 13, 2012
(86) PCT No.: PCT/US2012/064845

§ 371 Date: May 12, 2014

Related U.S. Application Data
(60) Provisional application No. 61/558,615, filed on Nov. 11, 2011.

(51) Int. Cl. A61K 35/74

(52) U.S. Cl. CPC A61K 35/747
USPC 424/93.45; 435/252.9

(57) ABSTRACT

Methods and compositions are hereby disclosed for enhancing weight gain and feed efficiency in a bird, such as a chicken, a turkey, or a laying hen, among others. The methods include administering to the bird a lactic acid producing bacterium (LAB) or combination of LABs. The disclosed methods and compositions also help reduce pathogen infection in the bird and reduce incidence of pathogen contamination in eggs produced by laying hens.

### RELATED APPLICATIONS

This application claims priority to U.S. Patent application 61/558,615 filed Nov. 11, 2011, the entire content of which is hereby incorporated by reference into this application.

### BACKGROUND

I. Field of the Invention

The present disclosure pertains to the use of one or more lactic acid producing bacteria (also referred to as “lactic acid bacteria” or “LAB” in this disclosure) to enhance the well being of an animal. More particularly, the disclosure relates to the use of lactic acid bacteria as a dietary supplement to improve feed efficiency and/or to reduce pathogen infection in the poultry industry.

II. Description of Related Art

Improving feed efficiency has been one of the objectives in the poultry industry. As the prices of feed and fuel increase, achieving higher feed efficiency is becoming even more important. Lactic acid producing bacteria have been shown to improve feed efficiency in ruminants. See, e.g., U.S. Pat. No. 5,534,271. However, no effects of lactic acid bacteria have been reported on birds, such as broilers. The digestive system of birds is substantially different from that of ruminants. The native microbial flora in birds are also very different from those of ruminants. Therefore, it is not known whether LAB supplement would improve the feed efficiency in birds.

In another aspect, maintaining a healthy stock is also a major concern for a poultry farm. Various pathogens are known to cause illnesses in birds. These diseases range from mild disorders to fatal diseases. In addition, certain pathogens may pose no significant harms to the birds but may pose extreme health risks for humans.

Pathogens may be passed from a bird to humans when humans get in contact with the bird or consume food products prepared from the bird. Food borne pathogen contamination may be controlled by minimizing contamination at several points of entry by pathogens.

U.S. Pat. No. 7,063,836 disclosed a unique combination of live lactic acid producing bacterium and live lactate utilizing bacterium as feed supplements (also known as direct-fed microbials (DFM) or probiotics) to help reduce pre-harvest infections in ruminants. The compositions and methods disclosed in U.S. Pat. No. 7,063,836 help reduce the numbers of enteropathogens such as E. coli O157:H7. By reducing the numbers of enteropathogens in animals that produce meat or milk, these methods help protect consumers of beef, dairy, and other food products from being infected by the pathogens. Because the structure of the digestive systems are different between ruminants and birds, and because ruminants and birds have different native microflora, it was not clear whether LAB supplementation would help reduce pathogen infection in poultry.

### SUMMARY

The present disclosure advances the art by providing methods and compositions for enhancing feed efficiency and for reducing pathogenic infection in an animal, such as a bird. In one aspect, the disclosed compositions and methods may be administered to a domesticated bird. Examples of birds may include but are not limited to a chicken, a laying hen, a duck, a goose, a turkey, a fowl, or a pheasant, among others. It is disclosed here that supplementing lactic acid producing bacteria (LAB) to a bird may enhance feed efficiency in the bird. In another aspect, administration of the lactic acid bacteria may help increase the yield of breast meat in the bird. In another aspect, the lactic acid bacteria may reduce infection of the bird by various pathogens, or reduce pathogen contamination of the carcass of the bird.

In one embodiment, the LAB may be fed to a laying hen as a dietary supplement to enhance the feed efficiency, to reduce pathogenic infection and to decrease the incidence of pathogens on the inside and/or outside of eggs produced by the hen. In one aspect, the LAB may be fed to the hen at a dosage that is sufficient to reduce the amount of at least one pathogen on the exterior surface of eggs produced by the hen by at least 30%, by about 60% or more, or by about 80% or more, as compared to the amount of said at least one pathogen on the exterior surface of eggs produced by an untreated bird. In another aspect, the dosage fed to the hen is sufficient to reduce the amount of at least one pathogen in the oviduct of the hen by at least 30%, by about 60% or more, or by about 80% or more, as compared to the amount of said at least one pathogen in the oviduct of an untreated bird. For purpose of this disclosure, the term “at least one pathogen” may include but not limited to one or more of Salmonella typhimurium, E. coli, Staphylococcus aureus and Campylobacter jejuni.

In one embodiment, the disclosed composition may contain one or more lactic acid producing bacteria (LAB). Examples of the LAB may include but are not limited to the genus of Lactobacillus. In one aspect, at least one of the lactic acid producing bacteria may be Lactobacillus acidophillus. Examples of Lactobacillus strains may include but are not limited to LA51, M35, LA45, NP28 (also known as C28) and L411 strains. In one aspect, more than one lactic acid producing bacteria that belong to the same or different species may be used in the supplement. In another aspect, the composition does not contain significant amount of lactic acid utilizing bacteria. As used here, “significant” means the intake of lactic acid utilizing bacteria via supplementation, if any, is less than 100 CFU per day. In another aspect, the composition does not contain lactic acid utilizing bacteria. Examples of lactic acid utilizing bacteria include but are not limited to Propionibacterium freudenreichii, among others.

In one embodiment, a method is disclosed for improving feed utilization in a bird wherein a composition comprising a Lactobacillus strain LA51 is administered to the bird at a dosage of from about 1×10³ to about 1×10¹⁰ CFU per day for each bird. In another embodiment, a method is disclosed for reducing pathogenic infection in a bird wherein a composition comprising a Lactobacillus strain LA51 is administered to the bird at a dosage of from about 1×10³ to about 1×10¹⁰ CFU per day for each bird. In another embodiment, a method is disclosed for reducing the amount of at least one pathogen on the exterior surface of eggs produced by a laying hen, wherein a composition comprising a Lactobacillus strain LA51 is administered to the hen at a dosage of from about 1×10³ to about 1×10¹⁰ CFU per day for each hen.

The lactic acid producing bacteria may be administered to the bird separately from regular feed and/or drinks. Alternatively, the bacteria may be administered to the bird along with regular feed and/or drinks. In one aspect, the lactic acid producing bacteria may be pre-mixed with feed or water and administered to the bird in the form of a pre-mix. In another aspect, the LAB may be pre-mixed with feed specific for domesticated birds, for example, feed specific for broiler chickens, before being administered to the birds.

Dosage of the lactic acid bacteria supplement may vary from species to species. The dosage may be determined based on factors such as body weight of the bird, stage of growth, or environmental conditions, among others. In one embodiment, one or more strains of lactic acid bacteria may be administered to the bird at a dosage of between 1×10³ and 1×10¹⁰ CFU for each strain per bird per day. In another aspect, the dosage is between 1×10³ and 1×10⁸ CFU for each strain per bird per day. In another aspect, the dosage is between 1×10⁴ and 1×10⁶ CFU for each strain per bird per day. In another aspect, the dosage is between 1×10⁶ and 1×10⁹ CFU for each strain per bird per day. In another aspect, the dosage is between 1×10⁷ and 1×10⁸ CFU for each strain per bird per day. In another aspect, the dosage is about 1×10⁵ CFU for each strain per bird per day. In another aspect, the dosage is about 1×10⁶ CFU for each strain per bird per day. In another aspect, the dosage is about 1×10⁷ CFU for each strain per bird per day.

The methods may include a step wherein all birds, or at least representatives of the birds, are assessed to determine if the birds are in need of LAB supplementation.

Before the disclosed composition is administered to a bird, the feed efficiency of the bird may be measured or predicted in order to determine if the bird is in need of lactic acid bacteria supplements. The term “feed efficiency” (also referred to as “feed conversion”) is defined as the amount of feed by pound consumed for each bird in order for that bird to gain one pound of weight in the case of all birds other than laying birds. In the case of laying hens, feed conversion is defined as the amount of feed by pound consumed for each bird in order for that bird to produce a pound of eggs. In some instances, kilogram may be used in place of pound as the measurement unit for weight. Feed efficiency may be calculated by dividing the feed intake by the weight gain during the same period. Alternatively, the inverse calculation may be used to calculate feed efficiency. Feed efficiency may fluctuate slightly depending on the different energy levels of different diets. For purpose of this disclosure, the calculation of feed efficiency is based on standard diets containing 3,000-3,200 kcal/kg in the starter, and up to about 3,100-3,300 kcal/kg in the finisher diet.

In one embodiment of this disclosure, during the first 21-30 days of life (namely, after hatching), the birds may be fed a starter diet containing between 3,000 and 3,200 kcal/kg of energy. In one aspect, the birds may be fed a starter diet containing about 3,100 kcal/kg of energy. In another aspect, the composition and energy levels of the starter, grower or finisher diets are provided herein for the purpose of illustration but not to be limiting. In another aspect, the specific selection of the composition and energy levels of the starter diets in combination with the specific dosages of the LAB disclosed herein may contribute to the improvements of feed efficiency and reduction of infection, among others.

In another embodiment, the birds may be fed a grower diet containing about 3,100-3,200 kcal/kg of energy during Day 20 to Day 40 of life. In one aspect, the grower diet may contain about 3,150 kcal/kg of energy. In another aspect, the specific selection of the composition and energy levels of the grower diets in combination with the specific dosages of the LAB disclosed herein may contribute to the improvements of feed efficiency and reduction of infection, among others.

In another embodiment, the birds may be fed a finisher diet containing about 3,100-3,300 kcal/kg of energy during Day 30 to Day 50 of life. In one aspect, the finisher diet may contain about 3,200 kcal/kg of energy. In another aspect, the specific selection of the composition and energy levels of the finisher diets in combination with the specific dosages of the LAB disclosed herein may contribute to the improvements of feed efficiency and reduction of infection, among others.

In one embodiment, if the measured or predicted feed efficiency for the first 21 days of life (from hatching) of a broiler is higher than 1.45, lactic acid bacteria supplement may be desired. In another embodiment, if the measured or predicted feed efficiency for the first 42 days of life (from hatching) is higher than 1.95, lactic acid bacteria supplement may be desired. After a period of supplements, the feed efficiency of the bird may be measured to determine the effects of the lactic acid bacteria supplements on feed efficiency. In one aspect, the lactic acid producing bacteria may help improve the feed efficiency of a bird by at least 2%, 3%, or 4%. In another aspect, the feed efficiency may be predicted based on empirical data obtained on same or similar breed of birds on same or similar feed and grown under same or similar conditions.

In another embodiment, the breast meat content of a bird fed with the lactic acid bacteria according to this disclosure is at least 1%, 2%, 3%, 6% or higher than that of a comparable bird fed with the same diet without the lactic acid bacteria supplement.

The disclosed method may include a step of (a) administering to a bird a supplement containing a lactic acid producing bacterium at a dosage of between 1×10³ and 1×10⁷ CFU of the LAB per day for each bird. In another aspect, the method may further include a step (b) of measuring the feed efficiency of the bird to determine if it is in need of the LAB supplement. Typically, step (b) is performed before said step (a). If it is determined that the bird is in need of the LAB supplement, step (a) is then performed.

The duration of the LAB supplementation varies. In one aspect, a broiler's diet may be supplemented with the LAB continuously for 20-60 days daily in order to achieve the desired effects. In another aspect, a turkey's diet may be supplemented with LAB continuously for 60-140 days daily, or for a period of about 80-120 days daily. Laying hens may require longer period of supplementation, for example, as long as 300 days, or longer. The LAB supplement is ideally provided to the bird continuously on a daily basis during the period of supplementation.

In another embodiment, the method may further include a step (c) to assess the effect of supplementation after at least 2 weeks of LAB supplementation performed in step (a). In one aspect, the feed efficiency obtained in step (c) is at least 2%, 3%, or 4% better than that obtained in step (b) described above. In another embodiment, breast meat content of the bird is determined or predicted in both step (b) prior to supplement and in step (c) after supplement. In one aspect, the breast meat content obtained in step (c) is at least 1% higher than that obtain in step (b).

In another embodiment, before the disclosed composition is administered to a bird, the health status of the bird may be measured or predicted to determine if the bird is in need of lactic acid bacteria supplements. In one aspect, mean lesion score in the intestine of the bird may be used as an indicator of the health status of a bird. If the measured or predicted intestinal lesion score is relatively high, lactic acid bacteria supplement may be needed. In another aspect, a mean lesion score of 0.5 or above may indicate that lactic acid bacteria supplement is desirable, or in other words, the birds are in need of lactic acid bacteria supplement. In another aspect, the health status of a bird may be predicted based on empirical data obtained on same or similar breed of birds on same or similar feed and grown under same or similar conditions. After a period of supplements, the health status of the bird may be measured to monitor the effects of the supplements on the health status of the bird.

Birds raised on built-up litter may be more susceptible to pathogen infection than birds raises on fresh litter. The disclosed lactic acid bacteria may be particularly effective in reducing pathogen infection in birds raised on built-up litter. In one aspect, the disclosed methods are suitable for commercial-type laying hen performance when placed under practical laying hen growout procedures. In another aspect, no antibiotic is fed to the birds when the birds while they are receiving the LAB supplements as disclosed herein. Common types of pathogens that may infection domesticated birds may include but are not limited to Salmonella, E. coli, Staphylococcus aureus and Campylobacter jejuni. Example of Salmonella may be Salmonella typhimurium species or other species of Salmonella. Example of E. coli may be E. coli O157:H7 strain or other pathogenic E. coli strains.

### DETAILED DESCRIPTION

This disclosure provides improved methods and compositions for enhancing the feed efficiency in birds. The disclosed methods and compositions may also help reduce pathogen infection in the birds.

As used herein, the term “pathogen” refers to a microorganism that may be harmful to a host animal, as well as a microorganism that may not be harmful to the host animal but may be harmful to a human who contacts with or consume the host animal or a product prepared from the host animal. By way of example, the most common pathogens in poultry include but are not limited to Salmonella spp. (e.g., Salmonella typhimurium), E. coli, Staphylococcus aureus and Campylobacter jejuni.

Various commercially available products are described or used in this disclosure. It is to be recognized that these products or associated trade names are cited for purpose of illustration only. Certain physical or chemical properties and composition of the products may be modified without departing from the spirit of the present disclosure. One of ordinary skill in the art may appreciate that under certain circumstances, it may be more desirable or more convenient to alter the physical and/or chemical characteristics or composition of one or more of these products in order to achieve the same or similar objectives as taught by this disclosure. It is to be recognized that certain products or organisms may be marketed under different trade names which may in fact be identical to the products or organisms described herein.

It is to be noted that, as used in this specification and the claims, the singular forms “a,” “an,” and “the” include plural referents unless the context clearly dictates otherwise. Thus, for example, reference to “a pathogen” includes reference to a mixture of two or more pathogens, reference to “a lactic acid producing bacterium” includes reference to one or more lactic acid producing bacteria.

The terms “between” and “at least” as used herein are inclusive. For example, a range of “between 5 and 10” means any amount equal to or greater than 5 but equal to or smaller than 10.

For purpose of this disclosure, the term “precede” means one event or step is started before a second event or step is started.

The dosage of the bacterial supplements is defined by “CFU per day,” which refers to the number of colony forming units of the particular bacterial strain that is administered on the days when the bacterial strain is administered.

The terms “untreated” and “unsupplemented” are used interchangeably, and refer to animals (or birds) that are fed identical or similar diet except for the omission of lactic acid producing bacteria from the diet. The term “performance” refers to one or more of the growth parameters, such as weight gain, feed conversion, and feed efficiency.

Administration of the bacterial supplement may be through oral ingestion with or without feed or water or may be mixed with feed and/or water. The bacterial supplement may be prepared as a pre-mix with feed and/or water or it may be mixed on site at the time of administration. In one aspect, the bacterial supplements are administered along with normal feed or water. In another aspect, the bacteria may be prepared in the form of a lyophilized culture before being mixed with water for spraying or blending with the feed and/or water. The final mixture may be in dry or wet form, and may contain additional carriers that are added to the normal feed of the birds. The normal feed may include one or more ingredients such as cereal grains, cereal grain by-products, or other commercial bird or poultry feed products. The lyophilized cultures may also be added to the drinking water of the birds.

Preparation of the bacterial supplement to be mixed with feed or water may be performed as described in U.S. Pat. No. 7,063,836. Detection and enumeration of pathogenic bacteria may be conducted as described in Stephens et al. (2007). The contents of these references are hereby expressly incorporated by reference into this disclosure.

In one embodiment, the lactic acid producing bacterium may include one or more of the following: Bacillus subtilis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidilactici, Pediococcus pentosaceus, Streptococcus cremoris, Streptococcus diacetylactis, Streptococcus (Enterococcus) faecium, Streptococcus intermedius, Streptococcus lactis, Streptococcus thermophilus, and combinations thereof.

Examples of lactate utilizing bacterium may include Megasphaera elsdenii, Peptostreptococcus asaccharolyticus, Propionibacterium freudenreichii, Propionibacterium acidipropionici, Propionibacterium globosum, Propionibacterium jensenii, Propionibacterium shermanii, Propionibacterium spp., Selenomonas ruminantium, and combinations thereof.

In one embodiment, the lactic acid producing bacterium is Lactobacillus acidophilus or Lactobacillus animalis. Examples of the lactic acid producing bacterium strains may include but are not limited to the LA51, M35, LA45, NP28, and L411. In another embodiment, the lactic acid producing bacterium strain is LA51. The term Lactobacillus acidophilus/animalis may be used to indicate that either Lactobacillus acidophilus or Lactobacillus animalis may be used. It is worth noting that when strain LA51 was first isolated, it was identified as a Lactobacillus acidophilus by using an identification method based on positive or negative reactions to an array of growth substrates and other compounds (e.g., API 50-CHL or Biolog test). Using modern genetic methods, however, strain LA51 has recently been identified as belonging to the species Lactobacillus animalis (unpublished results). Regardless of the possible taxonomic changes for LA51, the strain LA51 remains the same as the one that has been deposited with ATCC.

Lactobacillus strains C28, M35, LA45 and LA51 strains were deposited with the American Type Culture Collection (ATCC) on May 25, 2005 and have the Deposit numbers of PTA-6748, PTA-6751, PTA-6749 and PTA-6750, respectively. Lactobacillus strain L411 was deposited with the American Type Culture Collection (ATCC) on Jun. 30, 2005 and has the Deposit number PTA-6820.

The following examples are provided to illustrate the present disclosure, but are not intended to be limiting. The feed ingredients and supplements are presented as typical components, and various substitutions or modifications may be made in view of this disclosure by one of skills in the art without departing from the principle and spirit of the present invention.

Certain feeding tests described in the Examples contain ingredients that are in a size suitable for a small scale setting. It is important to note that these small scale tests may be scaled up and the principle of operation and the proportion of each ingredient in the system may equally apply to a larger scale feeding system. Unless otherwise specified, the percentages of ingredients used in this disclosure are on a w/w basis.

### Example 1

### LAB Supplement Improves Feed Efficiency and Breast Meat Content

This example describes the effect of lactic acid producing bacteria as feed supplements to market-age broiler chickens when reared on built-up litter as well as the effects of dose titration levels. This study was also conducted to determine the effective level to potentially improve live performance and meat processing criteria.

The test period began on the day of hatch of the chicks (Trial Day 0). The chicks were fed a commercial-type feed until the end of the study. Each of four (4) test treatments contained 12 replicates per treatment which was randomly assigned and each replicate contained 30 broilers for a total number of 1,440 animals in the study. Chicks were randomly assigned to treatments on Trial Day 0 (or day of hatching).

The chicks were observed daily for signs of unusual growout patterns or health problems. Body weights and food consumption were measured on trial days 21, 42 and 49. Mean body weight, feed conversion and mortality were also evaluated. Intestinal lesion scores (Day 14) were recorded. Other data collected included, for example, processing data and Litter Condition Scoring: termed Litter Condition Scores. Processing data may include, for example, the following measurements.
1. On Day 49, 10 males and 10 females from each pen were randomly selected, following a 9-11 hr feed withdrawal period, and dry yield (WOG or without giblets) was determined.
2. The fresh hot carcass was chilled for 1-2 hr and the large and small pectoral breast muscle yield was determined
3. Carcass Data Collection: dry yield %, total breast yield %, major pectoral %, and minor pectoral %.
4. All % data were calculated from both live weight and dry yield weight.

The lactic acid producing bacterium used in this study was Lactobacillus. Lactobacilli sources were used at various levels as specified below, based on per bird per day basis. These test materials were tested under a typical field stress condition with built-up litter from at least three previous flocks.

More specifically, a total of 1,600 mixed sex broiler chicks (a sufficient number to ensure availability of at least 1,440 healthy chicks for the study) were obtained from a commercial hatchery on Trial Day 0 (same as hatch date). These chicks were immediately transported to a Research Facility under temperature-controlled conditions to assure bird comfort. Animal care practices conforming to the Guide for the Care and Use of Agricultural Animals in Agricultural Research and Teaching (FASS, 1999) were used at all times.

Broilers were evaluated upon receipt for signs of disease or other complications that may have affected the outcome of the study. Following examination, broilers were weighed. Broilers were allocated to each pen and to treatment groups using a randomized block design. Weight distribution across the treatment groups was assessed prior to feeding by comparing the individual test and reference group standard deviations of the mean against that of the control group. Differences between control and test or reference groups were within one standard deviation, and as such, weight distribution across treatment groups was considered acceptable for this study.

There were 30 healthy/viable broilers per pen with 12 pens (replicates) per test group for a total of 360 broilers per treatment group. The broilers were fed ad libitum their respective treatment from time of hatching (termed in this study as Trial Day 0) to 42 days of age.

The broilers were housed in separated pens, with a 16″ high kick board between pens, located in a room containing forced air heaters with a cross-house ventilation system, precision controlled by the operation manager. Broilers were placed in 3.5′×9.5′ floor area and space with a minimum of 0.85 ft² per bird (without feeder and waterer space) provided. At least two nipple drinkers per pen (via well water) were used to provide water to the broilers.

Continuous (24 hr) incandescent lighting according to SOP lighting program for chicks was used during the entire study. Full lighting of >3 fc were used the first week and then dimmed to 1 fc for the next two weeks of age and then reduced to 0.5-0.8 fc during the remaining test period.

Diets for the birds were prepared as follows: Starter, Grower, and Finisher diets were formulated to meet minimum nutrient requirements of a typical commercial broiler diet using the NRC Nutrient Requirements for Poultry as a guideline (9^{th} edition, 1994). The nutrient values used for feed formulations conducted by a regression analysis program commonly used for Least-Cost Feed Formulation in the poultry industry are shown in Table 1. Briefly, feed formulations were conducted by a nutritionist by generating in a computer Least-Cost Feed Formulation program, using minimum nutrient level requirements (Published in NRC Nutrient Requirements for Poultry as a guideline (9th edition, 1994)) and by assuring a balance of all known nutrient requirements, using typical feed ingredients used in practical/commercial feed mills in the United States.

TABLE 1
Nutrient Values of Feed Formulations

| | Nutrient/Age | | |
|---|---|---|---|
| | Starter | Grower | Finisher |
| | 0-21 days | 22-35 days | 36-49 days |
| Metabolizable Energy (kcal/kg) | 3086 | 3142 | 3197 |
| Metabolizable Energy (kcal/#) | 1400 | 1425 | 1450 |
| Protein (%) | 21.00 | 20.00 | 18.00 |
| Lysine (%) | 1.20 | 1.12 | 1.08 |
| Methionine (%) Min | 0.50 | 0.45 | 0.40 |
| Methionine + Cystine (%) | 1.02 | 0.92 | 0.85 |
| Arginine (%) Min | 1.40 | 1.30 | 1.00 |
| Threonine (%) Min | 0.92 | 0.85 | 0.65 |
| Tryptophan (%) Min | 0.24 | 0.20 | 0.12 |
| Total Phosphorus (%) Min | 0.70 | 0.65 | 0.60 |
| Available Phosphorus (%) | 0.45 | 0.42 | 0.39 |
| Total Calcium (%) | 0.90 | 0.84 | 0.78 |
| Dietary Sodium (%) | 0.25 | 0.20 | 0.18 |
| Dietary Choline (g/kg) | 1.35 | 1.15 | 0.95 |

The chicks were fed the three different diets in three phases: Starter (Days 0 to 21), Grower (Days 22 to 35), and Finisher (Days 36 to 42). All diets were offered ad libitum. Fresh well water was also provided ad libitum.

The first mixing was conducted in a small plastic bag by adding 100 g corn oil and one pound of feed. Dietary protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production. Targeted ingredient compositions of Starter, Grower, and Finisher phase diets are presented in Table 1 above. Mixing equipment was flushed with ground corn prior to diet preparation. All diets were prepared using a paddle mixer. The mixer was cleaned between each diet (Starter, Grower, and Finisher) using compressed air and vacuum; mixing equipment was flushed with ground corn between each treatment group and flush material was retained for disposal.

Lactic acid bacteria (LAB), in this case, Lactobacillus acidophillus, were added to the diets via a series of Lactobacilli premixes. The feed was mixed every three to four days to assure that, on average, birds were receiving either 10⁴, 10⁵ or 10⁶ per bird per day. These Lactobacilli treatments were compared to a control, containing no added Lactobacilli and no other therapeutic or health additive.

The dosages of LABs for each test group are shown in Table 2 below. These LAB rations were fed to the broilers for a period of 42 days, with a common diet fed from Day 42-49 without any LAB supplements in the diets. Diets were fed in three phases in accordance with standard commercial poultry production practice: Starter (Days 0-21), Grower (Days 22-35), and Finisher (Days 36-49).

TABLE 2
Dosage of Lactic Acid Bacteria for Broiler

| | | | Lactobacilli |
|---|---|---|---|
| | | | Level |
| | | | (log number |
| Groups | Ration Number | Test Material (additive)^{1,2} | per bird)³ |
| T1 | NPC1-1 | None (no Lactobacilli | 0 |
| | | added) | |
| T2 | NPC1-2 | Lactobacilli Mixer #1 | Log 10⁴ per bird |
| T3 | NPC1-3 | Lactobacilli Mixer #2 | Log 10⁵ per bird |
| T4 | NPC1-4 | Lactobacilli Mixer #3 | Log 10⁶ per bird |

¹Control consisted of a normal broiler Starter, Grower and Finisher BASAL diets with no added Lactobacilli.
²Lactobacilli bacteria organisms were furnished by Nutrition Physiology Company, LLC.
³The calculation was made and 10% extra added to each premix (to assure that enough Lactobilli was administered to each bird).

The birds were observed at least three times daily for overall health, behavior and/or evidence of toxicity, and environmental conditions (results of Daily Observations). Temperature was checked in each pen three times daily.

No medications (other than treatment group test material) were administered during the feeding period. Mortalities were recorded and complete necropsy examinations were performed on all broilers found dead or moribund during the test period.

Live performance body weights and feed intakes were collected on Days 0, 21 and 42 during the test period. Weight gain, feed intake, and feed:gain ratio (feed efficiency) were calculated for Days 0-21, 22-42, 0-42, 43-49 and 0-49. Differences between broilers fed control and test groups were evaluated at P<0.05. Control group was CONTROL#1: no added Lactobacilli.

Mean body weight and body weight uniformity were measured at 0, 21, 42 and 49 days of age, and feed conversion calculations were performed for 0-21, 22-42, 0-42 and 43-49 days of age, respectively.

Processing data were also collected. On Day 49-50, 10 males and 10 females from each pen were randomly selected, following a 9-11 hr feed withdrawal period, and dry yield (WOG or without giblets) was determined. The fresh hot carcass was chilled for 1-2 hr and the large and small pectoral breast muscle yield was determined Carcass Data Collection: dry yield %, total breast yield %, major pectoral %, and minor pectoral %. All % data were calculated from both live weight and dry yield weight.

Carcasses of necropsied broilers and all birds remaining at the end of the study were disposed of according to local regulations via composting.

Table 3 shows the average body weight of T1-T4 groups chicks recorded on Day 0.

TABLE 3
Average Body Weight at Day 0

Treatment

| Criterion | T1 | T2 | T3 | T4 |
|---|---|---|---|---|
| Average Body Wt. (lb) Day 0 | 0.104 | 0.103 | 0.104 | 0.104 |
| Stat¹ | a | a | a | a |

¹Means within a row without a common superscript are significantly different (P < 0.05) as determined by Least Significant Difference.

Table 4 shows the average weight gain of T1-T4 groups at Day 21 and the respective Feed Conversion from Day 0 to Day 21 calculated based on weight gain and feed consumption.

TABLE 4
Weight Gain and Feed Conversion at Day 21

| | Treatment | | | |
|---|---|---|---|---|
| Criterion | T1 | T2 | T3 | T4 |
| Average Body Wt. (lb) Day 21 | 1.560 | 1.588 | 1.618 | 1.641 |
| Stat¹ | c | b | a | a |
| Feed Conversion Corrected Day 0-21 | 1.472 | 1.447 | 1.428 | 1.415 |
| Stat¹ | c | bc | b | a |
| Mortality % Day 0-21 | 0.96 | 0.64 | 0.96 | 0.96 |
| Stat¹ | a | a | a | a |
| Average Body Wt. Gain (lb) Day 0-21 | 1.455 | 1.484 | 1.513 | 1.536 |
| | c | b | a | a |

¹Means within a row without a common superscript are significantly different (P < 0.05) as determined by Least Significant Difference.

Table 5 shows the average weight gain of T1-T4 at Day 42 and the respective Feed Conversion from Day 0 to Day 42 calculated based on weight gain and feed consumption.

TABLE 5
Weight Gain and Feed Conversion at Day 42

| | Treatment | | | |
|---|---|---|---|---|
| Criterion | T1 | T2 | T3 | T4 |
| Average Body Wt. (lb) Day 42 | 4.689 | 4.772 | 4.872 | 4.880 |
| Stat¹ | c | b | a | a |
| Feed Conversion Corrected Day 0-42 | 1.965 | 1.922 | 1.906 | 1.888 |
| Stat¹ | c | bc | b | a |
| Mortality % Day 0-42 | 0.96 | 0.64 | 0.96 | 0.96 |
| Stat¹ | a | a | a | a |
| Average Body Wt. Gain (lb) Day 0-42 | 4.585 | 4.669 | 4.767 | 4.776 |
| | c | b | a | a |

¹Means within a row without a common superscript are significantly different (P < 0.05) as determined by Least Significant Difference.

Table 6 shows the average weight gain of T1-T4 from Day 21 to Day 42 and the respective Feed Conversion from Day 21 to Day 42 calculated based on weight gain and feed consumption.

TABLE 6
Feed Conversion from Day 21 to Day 42

| | Treatment | | | |
|---|---|---|---|---|
| Criterion | T1 | T2 | T3 | T4 |
| Feed Conversion corrected Day 21-42 | 2.215 | 2.163 | 2.147 | 2.130 |
| Stat¹ | b | a | a | a |
| Mortality % Day 21-42 | 0 | 0 | 0 | 0 |
| Stat¹ | a | a | a | a |
| Average Body Wt. Gain (lb) Day 21-42 | 3.130 | 3.185 | 3.254 | 3.240 |
| Stat¹ | b | ab | a | a |

¹Means within a row without a common superscript are significantly different (P < 0.05) as determined by Least Significant Difference.

The average body weight was also assessed at Day 49 and the feed conversion was calculated from Day 0 to Day 49 (Table 7).

TABLE 7
Body Weight at Day 49 and Feed Conversion Day 0-49

| | Treatment | | | |
|---|---|---|---|---|
| Criterion | T1 | T2 | T3 | T4 |
| Average Body Weight (lb) 49 day | 5.671 | 5.756 | 5.910 | 5.929 |
| Stat¹ | c | b | a | a |
| Feed Conversion Corrected: 0-49 days | 2.04 | 2.00 | 1.97 | 1.95 |
| Stat¹ | c | b | ab | a |
| Mortality (%): 0-49 days | 0.962 | 0.641 | 0.962 | 0.962 |
| Stat¹ | a | a | a | a |
| Average Body Weight Gain (period): Days | 5.567 | 5.653 | 5.806 | 5.825 |
| 0-49 | | | | |
| Stat¹ | c | b | a | a |

¹Means within a row without a common superscript are significantly different (P < 0.05) as determined by Least Significant Difference.

Table 8 shows the Feed Conversion from Day 43 to Day 49.

TABLE 8
Feed Conversion and Weight Gain from Day 43 to Day 49

| | Treatment | | | |
|---|---|---|---|---|
| Criterion | T1 | T2 | T3 | T4 |
| Feed Conversion Corrected: 43-49 days | 2.400 | 2.373 | 2.290 | 2.231 |
| Stat¹ | b | ab | ab | a |
| Mortality (%): 43-49 days | 0.00 | 0.00 | 0.00 | 0.00 |
| Stat¹ | a | a | a | a |
| Average Body Weight Gain (period): 43-49 | 0.982 | 0.984 | 1.038 | 1.049 |
| days | | | | |
| Stat¹ | a | a | a | a |

¹Means within a row without a common superscript are significantly different (P < 0.05) as determined by Least Significant Difference.

The yield of breast meat was also determined Table 9 shows significant improvement in total breast yield and major pectoral yield (live weight %) for 4 birds fed at least 10⁴ Lactobacilli when compared to the control group. There were no significant differences in minor pectoral yield (live weight %) among any of the treatments. Because separation of minor and major pectoral during removal from the body's bone structure is difficult to accomplish, the data on minor pectoral yield should be pooled with major pectoral to define potential statistical differences.

TABLE 9
Breast Meat Yield as Percentage of Live Weight

| | Treatment | | | |
|---|---|---|---|---|
| Criterion | T1 | T2 | T3 | T4 |
| Dry Yield (% WOG) | 69.493 | 69.736 | 69.860 | 70.154 |
| Stat¹ | c | bc | ab | a |
| Breast Yield (% live weight) | 16.54 | 16.64 | 16.78 | 16.87 |
| Stat¹ | d | c | b | a |
| Breast Yield (% WOG weight) | 23.817 | 23.878 | 24.029 | 24.064 |
| Stat¹ | b | b | a | a |
| Breast Major Pectorial Yield (% | 13.424 | 13.567 | 13.675 | 13.744 |
| live weight) | | | | |
| Stat¹ | c | b | ab | a |
| Breast Minor Pectorial Yield (% | 3.119 | 3.075 | 3.104 | 3.128 |
| live weight) | | | | |
| Stat¹ | a | a | a | a |

¹Means within a row without a common superscript are significantly different (P < 0.05) as determined by Least Significant Difference.

Table 10 shows significant improvement in total breast yield and major pectoral yield as percentage of WOG (without giblets) weight among the Groups supplemented with LABs.

TABLE 10
Breast Meat Yield as Percentage of WOG Weight

| | Treatment | | | |
|---|---|---|---|---|
| Criterion | T1 | T2 | T3 | T4 |
| Breast Major Pectoria | 19.327 | 19.467 | 19.581 | 19.601 |
| Yield (% WOG weight) | | | | |
| Stat¹ | b | ab | a | a |
| Breast Minor Pectorial | 4.49 | 4.41 | 4.45 | 4.45 |
| Yield (% WOG weight) | | | | |
| Stat¹ | a | a | a | a |
| Live 49-day Weight (g | 2681.029 | 2710.250 | 2745.029 | 2754.321 |
| of 20 birds processed) | | | | |
| Stat¹ | c | b | a | a |
| Dry Carcass Weight (g | 1866.450 | 1893.383 | 1921.438 | 1935.938 |
| of 20 birds processed) | | | | |
| Stat¹ | d | c | b | a |
| Breast Meat Weight (g | 444.746 | 452.354 | 461.896 | 465.904 |
| of 20 birds processed) | | | | |
| Stat¹ | d | c | b | a |

¹Means within a row without a common superscript are significantly different (P < 0.05) as determined by Least Significant Difference.

Litter Condition Scoring on each rearing pen was determined on Day 21 (at time of weighing) and Day 42 (end of study and following weighing procedures), according to the SOP. Litter Condition Score (for each pen) was determined using the following scale: 0=Litter balls up into a neat ball (without moisture being squeezed out), which is considered ideal litter moisture level. 1=Litter is too dry (Litter DOES NOT ball up into a neat ball). 2=Litter is too wet (Litter balls up, but moisture is squeezed out of the sample).

Overall, significant beneficial effects (P>0.05) were observed in weight gain and feed conversion (for both 21 and 42 days of age) when at least 10⁴ Lactobacilli per bird per day was included in the ration continuously during the grow-out period. Maximum live performance occurred at the 10⁶ Lactobacilli fed per bird per day. In general, with increasing levels of Lactobacilli, both weight gain and feed conversion improved. No differences were found in mortality and Litter Condition Score between broilers in rations containing an increased level of Lactobacilli. Based on the results from shown above, it may be concluded that the addition of Lactobacilli may improve broiler performance as compared to rations without the addition of Lactobacilli.

### Example 2

### LAB Supplements Reduce Lesion and Infection in Poultry

Intestinal lesion scores were also determined at 14 days of age for the T1-T4 groups of broilers as described above in Example 1. Special attention was paid to signs for coccidiosis and necrotic enteritis. Particular attention was also paid to any intestinal lining sloughing, redness, fragility or any other signs of intestinal damage.

Intestinal lesion scores were measured at 14 days of age (2 males and 2 females, including both coccidiosis signs and necrotic enteritis signs). As shown in Table 11, lesion scores were significantly improved with added levels of Lactobacilli over the control treatment. As Lactobacilli levels increased, feed conversion improved and lesion scores decreased. These results indicate that improved lesion scores with the use of higher levels of added Lactobacilli may improve broiler performance.

TABLE 11
Intestinal Lesion Scores

| | Treatment | | | |
|---|---|---|---|---|
| Criterion | T1 | T2 | T3 | T4 |
| Mean Lesion Score (14 days age) | 0.667 | 0.333 | 0.021 | 0.042 |
| Stat | c | b | a | a |

### Example 3

### Supplement of Lactic Acid Producing Bacterium to Laying Hens

This example describes studies carried out to assess the effect of lactic acid producing bacteria as feed supplements to laying hens. This study was also conducted to determine the effects of LAB on the quality of eggs produced by the treated hens. More specifically, these studies were carried out to determine the effect of Lactobacilli feed formula products and dose titration level on commercial egg-type layer live performance, egg parameter and egg production when reared in colony 3-bird cage system. Studies were also performed to determine if Lactobacilli may reduce the potential of Salmonella Incidence (presence/absence of Salmonella) and E. coli (Escherichia coli) contents in intestinal track fecal material and oviduct (that may contaminate the egg shell) as well as egg shell/egg content Salmonella spp. caused by feed/cage surface and feed contact.

A total of 210 commercial egg-type layers (a sufficient number to ensure availability of at least 180 healthy layers for the conduct of the study) were obtained from a commercial egg-type layer operation on Trial Days-21 (17-weeks of age). These were immediately transported to the Research Cage Units under temperature controlled conditions to assure bird comfort. After arrival at the research facility, layers were immediately randomized under the Standard Operating Procedures (or SOP). There were 3-healthy/viable commercial egg-type pullets per cage with 15 cages (replicates) per test group for a total of 45 commercial egg-type layers per treatment group. Commercial egg-type layers were housed three weeks, fed a prelay feed (with lower calcium levels) ad libitum for 3-weeks and fed their respective treatment from 20 weeks of age (termed in this Example as Trial Weeks 0) to 36 weeks of age (for four 28-day periods)

Animal care practices conformed to the Guide for the Care and Use of Agricultural Animals in Agricultural Research and Teaching (FASS, 1999). Commercial egg-type layers were obtained at 17-weeks of age from a commercial egg-type layer operation in Pennsylvania. Commercial egg-type layers were evaluated upon receipt for signs of disease or other complications that may have affected the outcome of the study. Following examination, commercial egg-type layers were weighed. Commercial egg-type layers were allocated to each cage and to treatment groups using a randomized block design. Weight distribution across the treatment groups was assessed prior to feeding by comparing the individual test and reference group standard deviations of the mean against that of the control group.

The test period began on Trial Day 0 (20-weeks of age), and layers were fed a commercial-type feed until the end of the study (for four 28-day periods). Each of four (4) test treatments contained 15 replicates per treatment (3-birds per replicate) randomly assigned and contained three (3) commercial egg-type layers per replicate for a total number of 45 animals on study. Layers were randomly assigned to treatments on Trial Weeks 0 (or 20-weeks of age).

A Lactobacillus bacterium (LA51 strain) was fed at the same daily rate per bird. Feed was mixed each three or four days to assure that, on average, birds were receiving either 10⁵, 10⁶ or 10⁷ CFU per bird per day. These Lactobacilli treatments were compared to a control, containing no added Lactobacilli and no other therapeutic or health additive. Stability was checked bi-weekly.

Commercial egg-type layers were housed in separated 3-bird colony cages, located in a room containing forced air heaters with a cross-house ventilation system, precision controlled by the operation manager. Commercial egg-type layers were placed in 12″×20″×18″ cage area and space with a required minimum of 67 in² per bird (without feeder and waterer space) provided. At least two nipple drinkers per cage (via well water) provided water to the birds.

LIGHTING PROGRAM: Incandescent lights using the SOP lighting program for commercial-type laying hens was employed. When lights are ON, lighting intensity was 2-3 fc. The lighting program employed was noted in the final report. In general, lighting was 16 hr of light (during daylight hrs, anticipating longest day of the year) and 8 hr of darkness. Lighting duration was not decreased so that the time of day was matched to maximum daylight during the entire test period and then only increased from there.

Commercial egg-type layers were observed at least three times daily for overall health, behavior and/or evidence of toxicity, and environmental conditions (results of Daily Observations). Temperature was checked in each cage three times daily. Drinking water and feed were provided ad libitum. No type of medication (other than treatment group test material) was administered during the entire feeding period. Mortalities (none were experienced in this trial) would have been recorded and complete necropsy examinations would have been performed on all commercial egg-type layers found dead or moribund.

During the prelay acclimation period and entire study, the pullets/commercial-type laying hens were observed daily for signs of unusual behavior patterns or health problems that was unique to caged animals. Such signs include but are not limited to cannibalism, feather picking, weak legs, “broodiness”, discolored/bleached-look waddles or combs and feet, and excessive body weight loss. Particular attention was paid to body weight gain/loss during the pre-trial acclimation period. The birds would be replaced if body weight gain was negative or minimum as compared to other flocks. Body weight change and food consumption were measured on Trial Days 28, 56, 84 and 112. Body weight and mortality were also evaluated and observations relative to egg production were recorded.

Prelay, Phase 1 Layer, Phase 2 Layer, Phase 3 and Phase 4 Layer diets were formulated to meet minimum nutrient requirements of a typical commercial egg-type layer diet using Feedstuffs, Reference Issue & Buyers Guide as a guideline (Vol 77, No. 38, 2006). The following Table 12 shows nutrient values used for feed formulations conducted by a regression analysis program commonly used for Least-Cost Feed Formulation in the commercial egg-type layer industry.

TABLE 12
Nutrient values of different feed formulations

| Dietary | | | | | |
|---|---|---|---|---|---|
| Nutrient | Prelay 17 | Phase 1 | Phase 2 | Phase 3 | Phase 4 |
| Composition | to 19 | 20 to 23 | 24 to 27 | 28 to 31 | 32 to 35 |
| Goal² | weeks¹ | weeks | weeks | weeks | weeks |
| ME (kcal/lb) | 1350₂ | 1330 | 1310 | 1295 | 1295 |
| ME (kcal/kg) | 2976 | 2932 | 2888 | 2855 | 2855 |
| Protein (%) | 14.50 | 15.50 | 16.76 | 16.76 | 16.76 |
| Calcium (%) | 2.50 | 3.55 | 3.56 | 3.56 | 3.56 |
| Avail. Phos. | 0.40 | 0.50 | 0.47 | 0.47 | 0.47 |
| (%) | | | | | |
| Sodium (%) | 0.18 | 0.23 | 0.19 | 0.19 | 0.19 |
| Chloride (%) | 0.16 | 0.22 | 0.22 | 0.22 | 0.22 |
| Potassium | 0.50 | 0.60 | 0.60 | 0.60 | 0.60 |
| (%) | | | | | |
| Arginine (%) | 0.90 | 1.15 | 1.15 | 1.15 | 1.15 |
| Lysine (%) | 0.70 | 0.95 | 0.84 | 0.84 | 0.84 |
| Methionine | 0.34 | 0.51 | 0.43 | 0.43 | 0.43 |
| (%) | | | | | |
| MET + CYS | 0.60 | 0.82 | 0.72 | 0.72 | 0.72 |
| (%) | | | | | |
| Threonine | 0.55 | 0.68 | 0.68 | 0.68 | 0.68 |
| (%) | | | | | |
| Tryptophan | 0.15 | 0.17 | 0.18 | 0.18 | 0.18 |
| (%) | | | | | |
| Linoleic | 1.0 | 1.5 | 1.5 | 1.5 | 1.5 |
| Acid (%) | | | | | |
| Dietary | 0.65 | 0.625 | 0.60 | 0.575 | 0.575 |
| Choline (%) | | | | | |

¹Ages represent approximate ages for each Phase of Production. Actual ages were 17-20 weeks for Pre-lay period, 21-24 weeks for Phase I, 25-28 weeks for Phase II, 29-32 weeks for Phase III, and 32-36 weeks for Phase IV.
²feed formulations were performed by a nutritionist by using a computer Least-Cost Feed Formulation program, using minimum nutrient level requirements (Published in Feedstuffs, Reference Issue & Buyers Guide as a guideline (Vol 77, No. 38, 2006) and assuring a balance of all known nutrient requirements, using typical feed ingredients used in practical/commercial feed mills in the USA. Feedstuff analyses are based on “as is” basis.

Lactobacilli were added to test diets via a series of Lactobacilli premixes added to each ration on an “as is” basis with first mixing (mixed biweekly) in a small plastic bag with the addition of 100 g corn oil and one (1) pound of feed. Dietary protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in commercial egg-type layer production. Targeted ingredient compositions of Prelay, Phase 1 Layer, Phase 2 Layer, Phase 3 Layer and Phase 4 Layer diets are presented in the table above. Mixing equipment was flushed with ground corn prior to diet preparation. All diets were prepared using a paddle mixer. The mixer was cleaned between each diet (Prelay, Phase 1 Layer, Phase 2 Layer, Phase 3 and Phase 4 Layer) using compressed air and vacuum; mixing equipment was flushed with ground corn between each treatment group and flush material was retained for disposal. The remaining corn was disposed of by composting at the facility.

Diets were fed in five phases: Prelay, Phase 1 Layer, Phase 2 Layer, Phase 3 and Phase 4 Layer. All diets were offered ad libitum. Fresh well water (from the research facility deep well) was provided ad libitum.

Rations of the supplements are shown in Table 13.

TABLE 13
Ration and Dosage of the LAB supplements for Laying Hens

| Ration | TEST MATERIAL | Lactobacilli Level |
|---|---|---|
| Number | (additive)_{1,2} | (cfu per bird per day)₄ |
| NPC3-1 | NONE (No added Lactobacillis) | None |
| NPC3-2 | Lactobacillis Mixer #1 | 10⁵ cfu/bird/day |
| NPC3-3 | Lactobacillis Mixer #2 | 10⁶ cfu/bird/day |
| NPC3-4 | Lactobacillis Mixer #3 | 10⁷ cfu/bird/day |

¹Control consisted of a normal egg-type layer Prelay, Phase 1 Layer, Phase 2 Layer, Phase 3 and Phase 4 Layer BASAL diets with no added Lactobacilli.
²The calculation was made and 10% extra added to each premix (to assure that enough Lactobacilli were administered per ton of feed.

These rations were fed to commercial egg-type layers (n=45/group, all females) for four 28-day periods. Diets were fed in three phases in accordance with standard commercial egg-type layer production practice as shown in Table 14.

TABLE 14
Feeding phases of LAB supplementation

| FEED | AGE of LAYERS | AGE | TRIAL DAYS |
|---|---|---|---|
| TYPE | (weeks of age)′ | (weeks of age)′ | RANGE |
| Prelay Ration | Week 17-20 | 17 weeks (time of | Trial Days −21 to |
| | | housing) to trial | Day 0 |
| | | initiation (Day 0)₁ | |
| Phase of | 20 to 24 weeks | 20 to 24 weeks | Trial Day 0-28 |
| production #1 | | | |
| Phase of | 25 to 28 weeks | 25 to 28 weeks | Trial Day 29-56 |
| production #2 | | | |
| Phase of | 29 to 32 weeks | 29 to 32 weeks | Trial Day 57-84 |
| production #3 | | | |
| Phase of | 33 to 36 weeks | 33 to 36 weeks | Trial Day 85-112 |
| production #4 | | | |

¹Actual age of layers

Stability of the Bacteria Premix was checked bi-weekly. No significant differences were found in stability. Table 15 shows a summary of the statistical effects of the LACTOBACILLI supplement on commercial-type laying hen performance when placed under practical laying hen growout procedures.

TABLE 15
Statistical effect of LACTOBACILLI PROBIOTICS On Commercial-
Type Laying Hen Performance When Placed Under Practical Laying Hen Growout
Procedures

| | Control vs. Treatment | Lactobacilli |
|---|---|---|
| | Significant | Level (Log# |
| Criterion | Not Significant (NS)¹ | cfu/per/day) |
| Albumen Weights Day 110-112 | NS | NS |
| Albumen Weights Day 26-28 | Significant | 10⁷ vs. 10⁵ |
| Albumen Weights Day 54-56 | Significant | 10⁷ vs. Control |
| Albumen Weights Day 82-84 | NS | NS |
| Bacteria: Bird Oviduct/Fecal E. coli (log10) Day 112 | Significant | 10⁷ vs. Control |
| Bacteria: Bird Oviduct/Fecal Sal. Incidence (%) D112 | Significant | 10⁷ vs. Control |
| Bacteria: Egg content E. coli (log10) Day 112 | NS | NS |
| Bacteria: Egg content E. coli (log10) Day 28 | NS | NS |
| Bacteria: Egg content E. coli (log10) Day 56 | NS | NS |
| Bacteria: Egg content E. coli (log10) Day 84 | NS | NS |
| Bacteria: Egg content Salmonella incidence Day 112 | NS | NS |
| Bacteria: Egg content Salmonella incidence Day 28 | NS | NS |
| Bacteria: Egg content Salmonella incidence Day 56 | NS | NS |
| Bacteria: Egg content Salmonella incidence Day 84 | NS | NS |
| Bacteria: Egg shell E. coli (log10) Day 112 | Significant | 10⁷ vs. 10⁵ |
| Bacteria: Egg shell E. coli (log10) Day 28 | NS | NS |
| Bacteria: Egg shell E. coli (log10) Day 56 | Significant | 10⁷ vs. Control |
| Bacteria: Egg shell E. coli (log10) Day 84 | Significant | 10⁷ vs. Control |
| Bacteria: Egg shell Salmonella incidence Day 112 | Significant | 10⁷ vs. 10⁵ |
| Bacteria: Egg shell Salmonella incidence Day 28 | Significant | 10⁷ vs. 10⁵ |
| Bacteria: Egg shell Salmonella incidence Day 56 | Significant | 10⁷ vs. 10⁵ |
| Bacteria: Egg shell Salmonella incidence Day 84 | Significant | 10⁷ vs. 10⁵ |
| Body Wt. (g) Day 0 | NS | NS |
| Body Wt. (g) Day 112 | Significant | 10⁷ vs. 10⁵ |
| Body Wt. (g) Day −14 | NS | NS |
| Body Wt. (g) Day 28 | NS | NS |
| Body Wt. (g) Day 56 | NS | NS |
| Body Wt. (g) Day 84 | Significant | 10⁷ vs. 10⁵ |
| Egg Cracks Day 110-112 | NS | NS |
| Egg Cracks Day 26-28 | NS | NS |
| Egg Cracks Day 54-56 | NS | NS |
| Egg Cracks Day 82-84 | NS | NS |
| Egg Grade Day 110-112 | Significant | 10⁷ vs. Control |
| Egg Grade Day 26-28 | NS | NS |
| Egg Grade Day 54-56 | NS | NS |
| Egg Grade Day 82-84 | NS | NS |
| Egg Production (%) Day 0-14 Bi-weekly | NS | NS |
| Egg Production (%) Day 0-28 | NS | NS |
| Egg Production (%) Day 14-28 Bi-weekly | NS | NS |
| Egg Production (%) Day 28-42 Bi-weekly | NS | NS |
| Egg Production (%) Day 28-56 | Significant | 10⁷ vs. Control |
| Egg Production (%) Day 42-56 Bi-weekly | Significant | 10⁷ vs. Control |
| Egg Production (%) Day 56-70 Bi-weekly | NS | NS |
| Egg Production (%) Day 56-84 | Significant | 10⁷ vs. 10⁵ |
| Egg Production (%) Day 70-84 Bi-weekly | Significant | 10⁷ vs. 10⁵ |
| Egg Production (%) Day 84-112 | Significant | 10⁷ vs. Control |
| Egg Production (%) Day 84-98 Bi-weekly | NS | NS |
| Egg Production (%) Day 98-112 Bi-weekly | Significant | 10⁷ vs. Control |
| Egg shell thickness (mm) Day 0-3 | NS | NS |
| Egg shell thickness (mm) Day 110-112 | NS | NS |
| Egg shell thickness (mm) Day 26-28 | NS | NS |
| Egg shell thickness (mm) Day 54-56 | NS | NS |
| Egg shell thickness (mm) Day 82-84 | NS | NS |
| Egg Weights Day 110-112 | NS | NS |
| Egg Weights Day 26-28 | Significant | 10⁷ vs. Control |
| Egg Weights Day 54-56 | Significant | 10⁷ vs. Control |
| Egg Weights Day 82-84 | NS | NS |
| Fat Pad (% of body weight) Day 112 | NS | NS |
| Feed Consumed (g/bird/day) Day 0-28 | NS | NS |
| Feed Consumed (g/bird/day) Day 28-56 | Significant | 10⁷ vs. Control |
| Feed Consumed (g/bird/day) Day 56-84 | NS | NS |
| Feed Consumed (g/bird/day) Day 84-112 | Significant | 10⁷ vs. Control |
| Feed Conversion (feed per dozen eggs) Day 0-28 | NS | NS |
| Feed Conversion (feed per dozen eggs) Day 28-56 | Significant | 10⁷ vs. Control |
| Feed Conversion (feed per dozen eggs) Day 56-84 | NS | NS |
| Feed Conversion (feed per dozen eggs) Day 84-112 | Significant | 10⁷ vs. Control |
| Feed Conversion (feed per egg) Day 28-56 | Significant | 10⁷ vs. Control |
| Feed Conversion (feed per egg) Day 56-84 | NS | NS |
| Feed Conversion (feed per egg) Day 84-112 | Significant | 10⁷ vs. Control |
| Feed Conversion (feed per kg egg) Day 0-112 | Significant | 10⁷ vs. 10⁵ |
| Feed Conversion (feed per kg egg) Day 28-56 | Significant | 10⁷ vs. Control |
| Feed Conversion (feed per kg egg) Day 56-84 | NS | NS |
| Feed Conversion (feed per kg egg) Day 84-112 | Significant | 10⁷ vs. Control |
| Feed Conversion (kg feed per egg) Day 0-28 | NS | NS |
| Feed Conversion (kg feed per kg egg) Day 0-28 | NS | NS |
| Haugh Units Day 110-112 | Significant | 10⁷ vs. Control |
| Haugh Units Day 26-28 | NS | NS |
| Haugh Units Day 36-42 | NS | NS |
| Haugh Units Day 54-56 | NS | NS |
| Haugh Units Day 64-70 | NS | NS |
| Haugh Units Day 8-14 | NS | NS |
| Haugh Units Day 82-84 | NS | NS |
| Haugh Units Day 92-98 | Significant | 10⁷ vs. Control |
| Intestinal lesion scores Day 112 | NS | NS |
| Mortality (%) Day 0-28 | NS | NS |
| Mortality (%) Day 28-56 | NS | NS |
| Mortality (%) Day 56-84 | NS | NS |
| Mortality (%) Day 84-112 | NS | NS |
| Shell Weights Day 110-112 | NS | NS |
| Shell Weights Day 26-28 | NS | NS |
| Shell Weights Day 54-56 | NS | NS |
| Shell Weights Day 82-84 | NS | NS |
| Specific gravity Day 110-112 | NS | NS |
| Specific gravity Day 26-28 | NS | NS |
| Specific gravity Day 36-42 | NS | NS |
| Specific gravity Day 54-56 | NS | NS |
| Specific gravity Day 64-70 | NS | NS |
| Specific gravity Day 8-14 | NS | NS |
| Specific gravity Day 82-84 | NS | NS |
| Specific gravity Day 92-98 | NS | NS |
| Yolk color (Lightness) Day 0-3 | NS | NS |
| Yolk color (Lightness) Day 110-112 | NS | NS |
| Yolk color (Lightness) Day 26-28 | NS | NS |
| Yolk color (Lightness) Day 54-56 | NS | NS |
| Yolk color (Lightness) Day 82-84 | NS | NS |
| Yolk color (redness) Day 0-3 | NS | NS |
| Yolk color (redness) Day 110-112 | NS | NS |
| Yolk color (redness) Day 26-28 | NS | NS |
| Yolk color (redness) Day 54-56 | NS | NS |
| Yolk color (redness) Day 82-84 | NS | NS |
| Yolk color (yellowness) Day 0-3 | NS | NS |
| Yolk color (yellowness) Day 110-112 | Significant | 10⁷ vs. 10⁵ |
| Yolk color (yellowness) Day 26-28 | Significant | 10⁷ vs. Control |
| Yolk color (yellowness) Day 54-56 | Significant | 10⁷ vs. 10⁵/Control |
| Yolk color (yellowness) Day 82-84 | Significant | 10⁷ vs. Control |
| Yolk Weights Day 110-112 | NS | NS |
| Yolk Weights Day 26-28 | NS | NS |
| Yolk Weights Day 54-56 | NS | NS |
| Yolk Weights Day 82-84 | NS | NS |

¹Significance (PR < 0.05) or deemed to be “statistically significant relationship” between treatment means and is defined as the P^{R}-value, using ANOVA for each criterion, is greater or equal to 0.0500

Tables 16, 17, 18 and 19 below show detailed data of the various effects of Lactobacillus probiotics on commercial-type laying hen when the birds are placed under practical laying hen growout procedure.

TABLE 16
Effect of Lactobacillus probiotics On Commercial-Type Laying Hen Performance
When Placed Under Practical Laying Hen Growout Procedures.

| | Treatment Data (cfu per bird per day) | | | |
|---|---|---|---|---|
| | Control | 1 × 10⁵ | 1 × 10⁶ | 1 × 10⁷ |
| Criterion | Mean Stat¹ | Mean Stat¹ | Mean Stat¹ | Mean Stat¹ |
| Albumen Weights Day 110-112 | 36.546^{a} | 36.518^{a} | 36.570^{a} | 36.626^{a} |
| Albumen Weights Day 26-28 | 32.769^{c} | 33.017^{bc} | 33.218^{ab} | 33.473^{a} |
| Albumen Weights Day 54-56 | 34.977^{b} | 35.221^{ab} | 35.167^{ab} | 35.326^{a} |
| Albumen Weights Day 82-84 | 36.067^{a} | 35.999^{a} | 36.002^{a} | 35.986^{a} |
| Bacteria: Bird Oviduct/Fecal E. coli (log10) Day 112 | 3.071^{b} | 3.013^{ab} | 2.998^{ab} | 2.931^{a} |
| Bacteria: Bird Oviduct/Fecal Sal. Incidence (%) D 112 | 33.333^{b} | 28.889^{ab} | 20.000^{ab} | 13.333^{a} |
| Bacteria: Egg content E. coli (log10) Day 112 | 0.088^{a} | 0.025^{a} | 0.069^{a} | 0.000^{a} |
| Bacteria: Egg content E. coli (log10) Day 28 | 0.000^{a} | 0.050^{a} | 0.045^{a} | 0.024^{a} |
| Bacteria: Egg content E. coli (log10) Day 56 | 0.038^{a} | 0.017^{a} | 0.059^{a} | 0.000^{a} |
| Bacteria: Egg content E. coli (log10) Day 84 | 0.064^{a} | 0.032^{a} | 0.019^{a} | 0.000^{a} |
| Bacteria: Egg content Salmonella incidence Day 112 | 1.111^{a} | 0.000^{a} | 0.000^{a} | 0.000^{a} |
| Bacteria: Egg content Salmonella incidence Day 28 | 3.016^{a} | 1.905^{a} | 0.000^{a} | 0.000^{a} |
| Bacteria: Egg content Salmonella incidence Day 56 | 0.952^{a} | 0.952^{a} | 0.000^{a} | 0.000^{a} |
| Bacteria: Egg content Salmonella incidence Day 84 | 2.857^{a} | 2.063^{a} | 0.000^{a} | 0.000^{a} |
| Bacteria: Egg shell E. coli (log10) Day 112 | 1.638^{ab} | 1.690^{b} | 1.524^{ab} | 1.416^{a} |
| Bacteria: Egg shell E. coli (log10) Day 28 | 1.351^{a} | 1.183^{a} | 1.368^{a} | 1.329^{a} |
| Bacteria: Egg shell E. coli (log10) Day 56 | 1.616^{b} | 1.579^{ab} | 1.410^{ab} | 1.261^{a} |
| Bacteria: Egg shell E. coli (log10) Day 84 | 1.596^{b} | 1.422^{ab} | 1.335^{ab} | 1.123^{a} |
| Bacteria: Egg shell Salmonella incidence Day 112 | 11.587^{b} | 10.952^{b} | 5.238^{ab} | 1.111^{a} |
| Bacteria: Egg shell Salmonella incidence Day 28 | 11.587^{b} | 12.222^{b} | 4.762^{ab} | 1.111^{a} |
| Bacteria: Egg shell Salmonella incidence Day 56 | 8.095^{b} | 8.095^{b} | 3.810^{ab} | 2.222^{a} |
| Bacteria: Egg shell Salmonella incidence Day 84 | 10.000^{b} | 11.111^{b} | 5.873^{ab} | 0.952^{a} |
| Body Wt. (g) Day 0 | 1338.940^{a} | 1319.669^{a} | 1316.809^{a} | 1346.589^{a} |
| Body Wt. (g) Day 112 | 1552.378^{b} | 1553.189^{b} | 1569.920^{ab} | 1606.009^{a} |
| Body Wt. (g) Day −14 | 1227.353^{a} | 1211.047^{a} | 1206.840^{a} | 1233.816^{a} |
| Body Wt. (g) Day 28 | 1448.982^{a} | 1430.491^{a} | 1425.227^{a} | 1458.256^{a} |
| Body Wt. (g) Day 56 | 1512.462^{a} | 1493.340^{a} | 1487.944^{a} | 1522.093^{a} |
| Body Wt. (g) Day 84 | 1533.729^{b} | 1526.258^{b} | 1542.076^{ab} | 1583.582^{a} |

¹Means within a row without a common superscript are significantly different (P < 0.05) as determined by Least Significant Difference.

TABLE 17
Effect of LACTOBACILLI PROBIOTICS On Commercial-Type Laying Hen Performance
When Placed Under Practical Laying Hen Growout Procedures.

| | Treatment Data (cfu per bird per day) | | | |
|---|---|---|---|---|
| | Control | 1 × 10⁵ | 1 × 10⁶ | 1 × 10⁷ |
| Criterion | Mean Stat¹ | Mean Stat¹ | Mean Stat¹ | Mean Stat¹ |
| Egg Cracks Day 110-112 | 0.085^{a} | 0.120^{a} | 0.053^{a} | 0.041^{a} |
| Egg Cracks Day 26-28 | 0.025^{a} | 0.033^{a} | 0.026^{a} | 0.022^{a} |
| Egg Cracks Day 54-56 | 0.044^{a} | 0.022^{a} | 0.030^{a} | 0.030^{a} |
| Egg Cracks Day 82-84 | 0.059^{a} | 0.037^{a} | 0.048^{a} | 0.071^{a} |
| Egg Grade Day 110-112 | 1.927^{b} | 2.000^{ab} | 2.009^{ab} | 2.056^{a} |
| Egg Grade Day 26-28 | 2.157^{a} | 2.142^{a} | 2.153^{a} | 2.116^{a} |
| Egg Grade Day 54-56 | 2.019^{a} | 2.084^{a} | 2.115^{a} | 2.103^{a} |
| Egg Grade Day 82-84 | 2.054^{a} | 2.078^{a} | 2.060^{a} | 2.054^{a} |
| Egg Production (%) Day 0-14 Bi-weekly | 65.238^{a} | 63.810^{a} | 59.524^{a} | 59.683^{a} |
| Egg Production (%) Day 0-28 | 77.540^{a} | 76.111^{a} | 74.365^{a} | 74.206^{a} |
| Egg Production (%) Day 14-28 Bi-weekly | 89.841^{a} | 88.413^{a} | 89.206^{a} | 88.730^{a} |
| Egg Production (%) Day 28-42 Bi-weekly | 93.175^{a} | 93.651^{a} | 94.286^{a} | 94.762^{a} |
| Egg Production (%) Day 28-56 | 93.492^{b} | 94.444^{ab} | 94.921^{ab} | 95.556^{a} |
| Egg Production (%) Day 42-56 Bi-weekly | 93.810^{b} | 95.238^{ab} | 95.556^{ab} | 96.349^{a} |
| Egg Production (%) Day 56-70 Bi-weekly | 95.556^{a} | 95.397^{a} | 96.667^{a} | 96.190^{a} |
| Egg Production (%) Day 56-84 | 94.444^{b} | 94.286^{b} | 96.111^{a} | 96.032^{a} |
| Egg Production (%) Day 70-84 Bi-weekly | 93.333^{b} | 93.175^{b} | 95.556^{a} | 95.873^{a} |
| Egg Production (%) Day 84-112 | 92.778^{b} | 93.651^{ab} | 94.048^{ab} | 94.524^{a} |
| Egg Production (%) Day 84-98 Bi-weekly | 93.651^{a} | 93.810^{a} | 93.968^{a} | 94.286^{a} |
| Egg Production (%) Day 98-112 Bi-weekly | 91.905^{b} | 93.492^{ab} | 94.127^{ab} | 94.762^{a} |
| Egg shell thickness (mm) Day 0-3 | 0.350^{a} | 0.348^{a} | 0.350^{a} | 0.351^{a} |
| Egg shell thickness (mm) Day 110-112 | 0.348^{a} | 0.349^{a} | 0.346^{a} | 0.346^{a} |
| Egg shell thickness (mm) Day 26-28 | 0.348^{a} | 0.348^{a} | 0.347^{a} | 0.347^{a} |
| Egg shell thickness (mm) Day 54-56 | 0.348^{a} | 0.348^{a} | 0.347^{a} | 0.348^{a} |
| Egg shell thickness (mm) Day 82-84 | 0.350^{a} | 0.347^{a} | 0.350^{a} | 0.347^{a} |
| Egg Weights Day 110-112 | 63.024^{a} | 63.095^{a} | 63.121^{a} | 63.090^{a} |
| Egg Weights Day 26-28 | 57.088^{b} | 57.246^{ab} | 57.574^{ab} | 57.658^{a} |
| Egg Weights Day 54-56 | 60.372^{b} | 60.611^{ab} | 60.720^{ab} | 60.757^{a} |
| Egg Weights Day 82-84 | 62.228^{a} | 62.074^{a} | 61.987^{a} | 62.109^{a} |
| Fat Pad (% of body weight) Day 112 | 1.047^{a} | 1.045^{a} | 1.055^{a} | 1.057^{a} |

¹Means within a row without a common superscript are significantly different (P < 0.05) as determined by Least Significant Difference

TABLE 18
Effect of LACTOBACILLI PROBIOTICS On Commercial-Type Laying Hen Performance
When Placed Under Practical Laying Hen Growout Procedures.

| | Treatment Data (cfu per bird per day) | | | |
|---|---|---|---|---|
| | Control | 1 × 10⁵ | 1 × 10⁶ | 1 × 10⁷ |
| Criterion | Mean Stat¹ | Mean Stat¹ | Mean Stat¹ | Mean Stat¹ |
| Feed Consumed (g/bird/day) Day 0-28 | 117.439^{a} | 115.781^{a} | 114.883^{a} | 115.166^{a} |
| Feed Consumed (g/bird/day) Day 28-56 | 125.122^{b} | 120.663^{ab} | 119.602^{a} | 117.401^{a} |
| Feed Consumed (g/bird/day) Day 56-84 | 127.075^{a} | 126.447^{a} | 124.749^{a} | 124.202^{a} |
| Feed Consumed (g/bird/day) Day 84-112 | 131.599^{b} | 129.742^{ab} | 129.024^{ab} | 126.718^{a} |
| Feed Conversion (feed per dozen eggs) Day 0-28 | 1.822^{a} | 1.830^{a} | 1.864^{a} | 1.871^{a} |
| Feed Conversion (feed per dozen eggs) Day 28-56 | 1.608^{b} | 1.534^{ab} | 1.513^{ab} | 1.476^{a} |
| Feed Conversion (feed per dozen eggs) Day 56-84 | 1.616^{a} | 1.610^{a} | 1.559^{a} | 1.553^{a} |
| Feed Conversion (feed per dozen eggs) Day 84-112 | 1.704^{b} | 1.663^{ab} | 1.646^{ab} | 1.609^{a} |
| Feed Conversion (feed per egg) Day 28-56 | 0.134^{b} | 0.128^{ab} | 0.126^{ab} | 0.123^{a} |
| Feed Conversion (feed per egg) Day 56-84 | 0.135^{a} | 0.134^{a} | 0.130^{a} | 0.129^{a} |
| Feed Conversion (feed per egg) Day 84-112 | 0.142^{b} | 0.139^{ab} | 0.137^{ab} | 0.134^{a} |
| Feed Conversion (feed per kg egg) Day 0-112 | 2.300^{c} | 2.256^{bc} | 2.226^{ab} | 2.197^{a} |
| Feed Conversion (feed per kg egg) Day 28-56 | 2.221^{b} | 2.109^{ab} | 2.077^{a} | 2.024^{a} |
| Feed Conversion (feed per kg egg) Day 56-84 | 2.164^{a} | 2.162^{a} | 2.096^{a} | 2.083^{a} |
| Feed Conversion (feed per kg egg) Day 84-112 | 2.253^{b} | 2.197^{ab} | 2.174^{ab} | 2.125^{a} |
| Feed Conversion (kg feed per egg) Day 0-28 | 0.152^{a} | 0.152^{a} | 0.155^{a} | 0.156^{a} |
| Feed Conversion (kg feed per kg egg) Day 0-28 | 2.659^{a} | 2.663^{a} | 2.698^{a} | 2.703^{a} |
| Haugh Units Day 110-112 | 63.679^{b} | 64.810^{ab} | 65.797^{ab} | 66.278^{a} |
| Haugh Units Day 26-28 | 67.756^{a} | 67.671^{a} | 66.310^{a} | 66.366^{a} |
| Haugh Units Day 36-42 | 65.644^{a} | 66.950^{a} | 67.252^{a} | 66.005^{a} |
| Haugh Units Day 54-56 | 65.940^{a} | 66.103^{a} | 67.303^{a} | 66.700^{a} |
| Haugh Units Day 64-70 | 65.287^{a} | 66.396^{a} | 65.548^{a} | 67.343^{a} |
| Haugh Units Day 8-14 | 67.963^{a} | 67.042^{a} | 68.400^{a} | 66.703^{a} |
| Haugh Units Day 82-84 | 65.918^{a} | 66.570^{a} | 66.906^{a} | 66.866^{a} |
| Haugh Units Day 92-98 | 65.266^{b} | 66.035^{ab} | 66.736^{ab} | 67.565^{a} |
| Intestinal lesion scores Day 112 | 1.356^{a} | 1.267^{a} | 1.333^{a} | 1.156^{a} |
| Mortality (%) Day 0-28 | 0.000^{a} | 0.000^{a} | 0.000^{a} | 0.000^{a} |
| Mortality (%) Day 28-56 | 0.000^{a} | 0.000^{a} | 0.000^{a} | 0.000^{a} |
| Mortality (%) Day 56-84 | 0.000^{a} | 0.000^{a} | 0.000^{a} | 0.000^{a} |
| Mortality (%) Day 84-112 | 0.000^{a} | 0.000^{a} | 0.000^{a} | 0.000^{a} |
| Shell Weights Day 110-112 | 7.002^{a} | 7.024^{a} | 6.930^{a} | 6.907^{a} |
| Shell Weights Day 26-28 | 6.331^{a} | 6.397^{a} | 6.332^{a} | 6.332^{a} |
| Shell Weights Day 54-56 | 6.663^{a} | 6.652^{a} | 6.673^{a} | 6.640^{a} |
| Shell Weights Day 82-84 | 6.877^{a} | 6.874^{a} | 6.798^{a} | 6.804^{a} |

¹Means within a row without a common superscript are significantly different (P < 0.05) as determined by Least Significant Difference

TABLE 19
Effect of LACTOBACILLI PROBIOTICS On Commercial-Type Laying Hen Performance
When Placed Under Practical Laying Hen Growout Procedures.

| | Treatment Data (cfu per bird per day) | | | |
|---|---|---|---|---|
| | Control | 1 × 10⁵ | 1 × 10⁶ | 1 × 10⁷ |
| Criterion | Mean Stat¹ | Mean Stat¹ | Mean Stat¹ | Mean Stat¹ |
| Specific gravity Day 110-112 | 1.083^{a} | 1.083^{a} | 1.083^{a} | 1.083^{a} |
| Specific gravity Day 26-28 | 1.083^{a} | 1.084^{a} | 1.084^{a} | 1.084^{a} |
| Specific gravity Day 36-42 | 1.084^{a} | 1.084^{a} | 1.083^{a} | 1.083^{a} |
| Specific gravity Day 54-56 | 1.084^{a} | 1.083^{a} | 1.084^{a} | 1.084^{a} |
| Specific gravity Day 64-70 | 1.085^{a} | 1.084^{a} | 1.083^{a} | 1.084^{a} |
| Specific gravity Day 8-14 | 1.084^{a} | 1.084^{a} | 1.084^{a} | 1.084^{a} |
| Specific gravity Day 82-84 | 1.084^{a} | 1.083^{a} | 1.083^{a} | 1.084^{a} |
| Specific gravity Day 92-98 | 1.083^{a} | 1.083^{a} | 1.085^{a} | 1.084^{a} |
| Yolk color (Lightness) Day 0-3 | 56.857^{a} | 56.812^{a} | 56.868^{a} | 56.789^{a} |
| Yolk color (Lightness) Day 110-112 | 56.938^{a} | 56.933^{a} | 56.940^{a} | 56.784^{a} |
| Yolk color (Lightness) Day 26-28 | 56.801^{a} | 56.801^{a} | 56.814^{a} | 56.922^{a} |
| Yolk color (Lightness) Day 54-56 | 56.881^{a} | 56.872^{a} | 56.810^{a} | 56.757^{a} |
| Yolk color (Lightness) Day 82-84 | 56.752^{a} | 56.847^{a} | 56.868^{a} | 56.796^{a} |
| Yolk color (redness) Day 0-3 | −13.714^{a} | −13.800^{a} | −13.824^{a} | −13.727^{a} |
| Yolk color (redness) Day 110-112 | −13.683^{a} | −13.759^{a} | −13.776^{a} | −13.682^{a} |
| Yolk color (redness) Day 26-28 | −13.711^{a} | −13.819^{a} | −13.723^{a} | −13.740^{a} |
| Yolk color (redness) Day 54-56 | −13.691^{a} | −13.749^{a} | −13.788^{a} | −13.712^{a} |
| Yolk color (redness) Day 82-84 | −13.648^{a} | −13.722^{a} | −13.781^{a} | −13.780^{a} |
| Yolk color (yellowness) Day 0-3 | 52.745^{a} | 52.539^{a} | 52.779^{a} | 52.566^{a} |
| Yolk color (yellowness) Day 110-112 | 52.421^{b} | 52.509^{b} | 52.754^{a} | 52.866^{a} |
| Yolk color (yellowness) Day 26-28 | 52.569^{b} | 52.682^{ab} | 52.709^{ab} | 52.741^{a} |
| Yolk color (yellowness) Day 54-56 | 52.514^{bc} | 52.494^{c} | 52.683^{ab} | 52.737^{a} |
| Yolk color (yellowness) Day 82-84 | 52.511^{b} | 52.557^{ab} | 52.677^{ab} | 52.713^{a} |
| Yolk Weights Day 110-112 | 19.477^{a} | 19.553^{a} | 19.621^{a} | 19.557^{a} |
| Yolk Weights Day 26-28 | 17.989^{a} | 17.832^{a} | 18.025^{a} | 17.852^{a} |
| Yolk Weights Day 54-56 | 18.732^{a} | 18.738^{a} | 18.880^{a} | 18.792^{a} |
| Yolk Weights Day 82-84 | 19.283^{a} | 19.201^{a} | 19.188^{a} | 19.319^{a} |

¹Means within a row without a common superscript are significantly different (P < 0.05) as determined by Least Significant Difference

Body Weight, Feed Consumed and Feed Conversion Results: Significant differences (P<0.05) were observed in mean body weight between 10⁵ and 10⁷ Lactobacilli fed levels on Days 84 and 112. The level of 10⁶ and 10⁷ were NOT significantly different (P>0.05). No significant differences (P>0.05) were found among treatments prior to Day 84.

Significant differences (P<0.05) were observed in mean feed conversion (kg feed per kg egg) between 10⁵ and 10⁷ Lactobacilli fed levels on Day 112. The level of 10⁶ and 10⁷ were NOT significantly different (P>0.05). Additionally, other feed conversion criteria were NOT significantly different (P>0.05) between 10⁵ and 10⁷ Lactobacilli fed levels at these time points.

Significant differences (P<0.05) were observed in feed conversion criteria (feed consumed, kg feed per dozen eggs, kg feed per egg, and kg feed per kg egg) between Control (with no added Lactobacilli) and 10⁷ Lactobacilli fed levels on Days 28-56 and 84-112, but were not significantly different at other days of production. The level of 10⁵, 10⁶ and 10⁷ were NOT significantly different (P>0.05) at these time points.

Egg Shell E. coli Bacteria Results: Significant differences (P<0.05) were observed in mean egg shell E. coli bacteria between 10⁵ and 10⁷ Lactobacilli fed levels on Day 112. The level of 10⁶ and 10⁷ were NOT significantly different (P>0.05) at these time points.

Significant differences (P<0.05) were observed in mean egg shell E. coli bacteria criteria between Control (with no added Lactobacilli) and 10⁷ Lactobacilli fed levels on Days 56 and 84. The level of 10⁵, 10⁶ and 10⁷ were NOT significantly different (P>0.05) at these time points.

No significant differences (P>0.05) were found in mean egg shell E. coli bacteria among all treatments prior to Day 56.

Egg Shell Salmonella Incidence Bacteria Results: Significant differences (P<0.05) were observed in mean egg shell Salmonella spp. bacteria incidence between 10⁵ and 10⁷ Lactobacilli fed levels on Days 28, 56, 84 and 112. These represented all days tested from the trial initiation. The level of 10⁶ and 10⁷ were NOT significantly different (P>0.05) at these time points.

Oviduct/Fecal E. coli and Salmonella Incidence Bacteria Results: Significant differences (P<0.05) were observed in mean Oviduct/Fecal E. coli and Salmonella spp. Incidence bacteria criteria between Control (with no added Lactobacilli) and 10⁷ Lactobacilli fed levels on Day 112. The level of 10⁵, 10⁶ and 10⁷ were NOT significantly different (P>0.05) at these time points. Day 112 was the only time tested during the trial.

Layer Mortality and Intestinal Lesion Score Results: No significant differences (P>0.05) were found among treatments on all trial days tested in mortality and intestinal lesion score (Day 112).

Egg Weight Measurement Results: Significant differences (P<0.05) were observed in mean egg weights between Control (with no added Lactobacilli) and 10⁷ Lactobacilli fed levels on Days 26-28 and 54-56, but were not significantly different at other days of egg production. The level of 10⁵, 10⁶ and 10⁷ were NOT significantly different (P>0.05) at these time points.

Albumen Weights Results: Significant differences (P<0.05) were observed in mean albumen weights between 10⁵ and 10⁷ Lactobacilli fed levels on Days 26-28. On Days 26-28, the level of 10⁶ and 10⁷ were NOT significantly different (P>0.05) at these time points. Significant differences (P<0.05) were observed in albumen weights criteria between Control (with no added Lactobacilli) and 10⁷ Lactobacilli fed levels on Days 26-28. No significant differences (P>0.05) in mean albumen weights were found among treatments on other trial days tested.

Egg Production Results: Significant differences (P<0.05) were observed in mean Egg Production (%) between 10⁵ and 10⁷ Lactobacilli fed levels on Days 56-84 and 70-84 biweekly data. On these same trial days, the level of 10⁶ and 10⁷ were NOT significantly different (P>0.05). Significant differences (P<0.05) were observed in Egg Production (%) criteria between Control (with no added Lactobacilli) and 10⁷ Lactobacilli fed levels on Days 28-56, 84-112, 42-56 biweekly, and 98-112 biweekly data. No significant differences (P>0.05) in Egg Production (%) were found among treatments on other trial days tested.

Egg Quality Measurement Results: Significant differences (P<0.05) were observed in mean Yolk Yellowness Color between 10⁵ and 10⁷ Lactobacilli fed levels on Days 110-112 data. On these same trial days, the level of 10⁶ and 10⁷ were NOT significantly different (P>0.05). No significant differences (P>0.05) in Yolk Yellowness Color were found among treatments on other trial days tested.

Significant differences (P<0.05) were observed in Yolk Yellowness Color between Control (with no added Lactobacilli) and 10⁷ Lactobacilli fed levels on Days 26-28, 54-56 and 82-84, but no significant differences were observed at other days of egg production. The level of 10⁵, 10⁶ and 10⁷ were NOT significantly different (P>0.05) at these time points.

Significant differences (P<0.05) were observed in mean Haugh Units measurement between Control (with no added Lactobacilli) and 10⁷ Lactobacilli fed levels on Days 92-98 and 110-112, but were not significantly different at other days of egg production. The level of 10⁵, 10⁶ and 10⁷ were NOT significantly different (P>0.05). No significant differences (P>0.05) in Haugh Units were found among treatments on other trial days tested.

Significant differences (P<0.05) were observed in Egg Grade between Control (with no added Lactobacilli) and 10⁷ Lactobacilli fed levels on Days 110-112 (representing the last days of the trial), but were not significantly different at other days of egg production. The level of 10⁵, 10⁶ and 10⁷ were NOT significantly different (P>0.05).

No significant differences (P>0.05) were found among treatments on all trial days tested in mean egg cracks (%), egg shell thickness (mm), yolk color (Lightness), yolk color (redness), shell weights, yolk weights, specific gravity, egg grade or mortality.

In summary, these results show that feeding Lactobacilli affect body weight maintenance (after 84 days on trial), feed conversion (after 28 days on trial), egg quality parameters (after 28 days on trial) and egg production (after 28 days on trial). These data also suggest that it is advisable that commercial egg-type layer industry use at least 10⁶ cfu of supplemented Lactobacilli per bird per day in all rations fed to commercial egg-type layers. An increased dosage at 10⁷ cfu per bird per day of added Lactobacilli may lead to improved effects on either or both the laying hens and the eggs. According to the present disclosure, Lactobacilli bacteria are stable when feeds are mixed bi-weekly and placed into the laying hen house where birds are housed. The incidence of E. coli and Salmonella spp. is reduced with increasing levels of Lactobacilli fed to laying hens. E. coli counts (cfu's, or Colony Forming Units, per ml of carcass rinse solution) and Salmonella incidence (% of commercial egg-type layers per cage) of processed carcasses are significantly improved with at least 10⁶ cfu per bird per day Lactobacilli over the control treatment.

### Example 4

### Supplement of Lactic Acid Producing Bacterium to Turkey

This Example shows the results of a study conducted to determine the effect of Lactobacilli feed formula products and dose titration level on large-bird market age turkey male (Meleagris gallopavo) performance when reared on built-up litter, as well as to determine if Lactobacilli may reduce the potential of Salmonella incidence (presence/absence of Salmonella) and E. coli contents in whole-bird rinse samples taken after complete processing.

The test period began on Trial Day 0 (day of hatch of poults), and poults were fed a commercial-type feed with or without the different dosages of supplements until the end of the study. Each of four (4) test treatments contained 12 replicates per treatment randomly assigned and contained 18 male turkeys per replicate for a total number of 864 animals on study. Poults were randomly assigned to treatments on Trial Day 0 (or day of hatch).

The poults were observed daily for signs of unusual growout patterns or health problems. Mean body weights were measured on trial days 0, 42 and 84. Feed consumption was measured on trial days 0, 21, 42, and 84. Intestinal lesion scores (Day 21) were recorded. Other data collected included the following:

Processing Data: (1) On Day 85-86, all remaining birds from each pen were processed, following a 9-11 hour feed withdrawal period, and dry yield (WOG or without giblets) was determined; (2) The fresh hot carcass was chilled overnight and the large and small pectoral breast muscle yield was determined; (3) Carcass Data Collection: dry yield %, total breast yield %, major pectoral %, and minor pectoral %; (4) All % was calculated from both live weight and dry yield weight. Litter Condition Scoring was also performed to obtain the Litter Condition Scores.

Lactobacilli sources were used at various levels, based on per bird per day basis. These test materials were tested under a typical field stress condition with built-up litter from at least three previous flocks. As shown in Table 20, a Lactobacilli source was fed at the same daily rate per bird at various dosages and negative controls. Feed was mixed each three to four days to assure that, on average, birds were receiving either 10⁵, 10⁶ or 10⁷ per bird per day. These Lactobacilli supplements were compared to a control, containing no added Lactobacilli and no other therapeutic or health additive. Stability of the Lactobacilli supplements was checked bi-weekly.

TABLE 20
Rations of Lactobacilli Supplements for Turkey

| | Ration | TEST MATERIAL | Lactobacilli Level |
|---|---|---|---|
| | Number | (additive) 1, 2, 3, 4 | (cfu per bird) 4 |
| T1 | NPC2-1 | NONE (No added Lactobacillis) | None |
| T2 | NPC2-2 | Lactobacillis Mixer #1 | 10⁵ cfu/bird |
| T3 | NPC2-3 | Lactobacillis Mixer #2 | 10⁶ cfu/bird |
| T4 | NPC2-4 | Lactobacillis Mixer #3 | 10⁷ cfu/bird |

1 Control consisted of a normal turkey male Starter, Grower and Finisher BASAL diets with no added Lactobacilli.
2 Lactobacilli bacteria organisms furnished by a commercial source. added Lactobacilli.
3 The calculation was made and 10% extra added to each premix (to assure that enough Lactobacilli were administered per ton of feed.
4 Stability was checked bi-weekly.

These rations were fed to Nicholas tom poults (n=216/group, all male) for a period of 84 days. Diets were fed in three phases in accordance with standard commercial turkey production practice: Starter (Days 0-21), Grower (Days 22-42), and Finisher (Days 43-84).

The commercial-simulated test model employed in this study used male turkey poults reared to a normal turkey industry age (84 days of age) at a normal floor space requirement (minimum of 3.33 ft² per bird), reared on used built-up litter from previous flocks. Rations formulations were conducted via computer-generated linear regression program that simulates formulations conducted during practical turkey production techniques. Treatments were tested in male turkeys, using pine shaving built-up litter floor experimental units. Turkeys were fed their experimental diets from time of placement (Day 0 immediately after hatch) to 84 days of age.

Turkey poults were randomized and housed into each pen onto floor pens. Each pen had sufficient floor, feeder and waterer space for each growout pen area. Following 84 days of growout, turkeys were weighed, feed consumption determined, and feed conversion (feed consumed/body weight) calculated.

A total of 900 male turkey poults (a sufficient number to ensure availability of at least 864 healthy poults for the conduct of the study) were obtained from a commercial hatchery on Trial Day 0 (same as hatch date). These were immediately transported to the research Facility under temperature-controlled conditions to assure bird comfort. After arrival at the research facility, poults were immediately randomized under Standard Operating Procedures (or SOP) of the Facility. There were 18 healthy/viable turkeys per pen with 12 pens (replicates) per test group for a total of 216 turkeys per treatment group. Turkeys were fed ad libitum their respective treatment from time of hatching (termed in this study as Trial Day 0) to 84 days of age.

Animal care practices conformed to the Guide for the Care and Use of Agricultural Animals in Agricultural Research and Teaching (FASS, 1999). Commercial turkeys (Nicholas Strain) were obtained at hatch (Trial Day 0) from a commercial hatchery. Turkeys were evaluated upon receipt for signs of disease or other complications that may have affected the outcome of the study. Following examination, turkeys were weighed. Turkeys were allocated to each pen and to treatment groups using a randomized block design. Weight distribution across the treatment groups was assessed prior to feeding by comparing the individual test and reference group standard deviations of the mean against that of the control group. Differences between control and test or reference groups were within one standard deviation, and as such, weight distribution across treatment groups was considered acceptable for this study.

Turkeys were housed in separated pens, with a 16″ high kick board between pens, located in a room containing forced air heaters with a cross-house ventilation system, precision controlled by the operation manager. Turkeys were placed in 5′×12′ floor area and space with a minimum of 3.33 ft² per bird (without feeder and waterer space) provided. At least two nipple drinkers per pen (via well water) provided water.

Continuous (24 hr) use of incandescent lights, using SOP lighting program for poults, was used during the entire study. Full lighting of 3-4 fc were used the first 10 days and then dimmed to 1 fc for the remainder of the test period.

Turkeys were observed at least three times daily for overall health, behavior and/or evidence of toxicity, and environmental conditions (results of Daily Observations). Temperature was checked in each pen three times daily. Drinking water and feed were provided ad libitum.

No type of medication (other than treatment group test material) was administered during the entire feeding period. Mortalities were recorded and complete necropsy examinations were performed on all turkeys found dead or moribund.

Live performance body weights and feed intakes were collected on Days 0, 42 and 84 during the growing period. Weight gain, feed intake, and feed:gain ratio (feed efficiency) were calculated for Days 0-42, 43-84 and 0-84. Differences between turkeys fed control and test groups were evaluated at P<0.05. Control group was considered to be the following: CONTROL#1: no added Lactobacilli.

Other data collected included the following: (1) Bacteria premix stability was checked bi-weekly; (2) Intestinal lesion scores (21 days of age, especially for coccidiosis and necrotic enteritis signs, particular attention was paid to any intestinal lining sloughing, redness, fragility or any other signs of intestinal damage); (3) Processing Data, (4) Litter Condition Scoring: Litter Scores; (5) E. coli counts (CFU's/ml rinse solution); (6) Salmonella incidence (%).

Processing Data included the following: (1) On Day 85-86, all remaining turkeys from each pen underwent a 9-11 hr feed withdrawal period, and dry yield (WOG or without giblets) was determined; (2) The fresh hot carcass was chilled overnight and the large and small pectoral breast muscle yield was determined; (3) Carcass Data Collection: dry yield %, total breast yield %, major pectoral %, and minor pectoral %; (4) All % was calculated from both live weight and dry yield weight.

Carcasses of necropsied turkeys and all birds remaining at the end of the study were disposed of according to local regulations via composting.

Starter, Grower, and Finisher diets were formulated to meet minimum nutrient requirements of a typical commercial turkey diet using Feedstuffs, Reference Issue & Buyers Guide as a guideline (Vol 77, No. 38, 2006). Table 21 shows nutrient values used for feed formulations conducted by a regression analysis program commonly used for Least-Cost Feed Formulation in the turkey industry.

TABLE 21
Nutrient values used for feed formulations

| | Age | | |
|---|---|---|---|
| | Starter | Grower | Finisher |
| Nutrient | 0-21 days | 22-42 day* | 45-84 day* |
| Metabolizable Energy (kcal/ kg) | 2866 | 2976 | 3086 |
| Metabolizable Energy (kcal/#) | 1300 | 1350 | 1400 |
| Protein (%) | 28.00 | 26.00 | 23.00 |
| Lysine (%) | 1.70 | 1.60 | 1.45 |
| Methionine (%) Min | 0.62 | 0.56 | 0.52 |
| Methionine + Cystine (%) | 1.05 | 0.93 | 0.84 |
| Arginine (%) Min | 1.75 | 1.65 | 1.55 |
| Threonine (%) Min | 0.90 | 0.87 | 0.82 |
| Tryptophan (%) Min | 0.28 | 0.26 | 0.23 |
| Total Phosphorus (%) Min | 0.75 | 0.70 | 0.65 |
| Available Phosphorus (%) | 0.75 | 0.70 | 0.65 |
| Total Calcium (%) | 1.40 | 1.25 | 1.15 |
| Dietary Sodium (%) | 0.20 | 0.18 | 0.18 |
| Dietary Choline (g/kg) | 1.60 | 1.40 | 1.10 |

¹ A Nutritionist conducted feed formulations, generated in a computer Least-Cost Feed Formulation program, using minimum nutrient level requirements (Published in Feedstuffs, Reference Issue & Buyers Guide as a guideline (Vol77, No. 38, 2006)) and assuring a balance of all known nutrient requirements, using typical feed ingredients used in practical/commercial feed mills in the USA. Feedstuff analyses are based on “as is” basis.

Lactobacilli were added to test diets via a series of Lactobacilli premixes furnished by a commercial source, added to each ration on an “as is” basis with first mixing in a small plastic bag with the addition of 100 g corn oil and one (1) pound of feed. Dietary protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in turkey production. Targeted ingredient compositions of Starter, Grower, and Finisher phase diets are presented in Table 21. Mixing equipment was flushed with ground corn prior to diet preparation. All diets were prepared using a paddle mixer. The mixer was cleaned between each diet (Starter, Grower, and Finisher) using compressed air and vacuum; mixing equipment was flushed with ground corn between each treatment group and flush material was retained for disposal. The remaining corn was disposed of by composting at the research facility.

Diets were fed in three phases: Starter (Days 0 to 21), Grower (Days 22 to 42), and Finisher (Days 43 to 84). All diets were offered ad libitum. Fresh well water (from the research facility deep well) was provided ad libitum. Mean body weight, body weight uniformity, and feed conversion calculations were performed for 0-42, 43-84, and 0-84 days of age. Intestinal lesion scores were performed at 21 days of age (2 males per pen, including both coccidiosis signs and necrotic enteritis signs).

Litter Condition Scoring on each rearing pen was determined on Day 21 (at time of weighing), Day 42, and Day 84 (end of study and following weighing procedures), according to SOP of the Facility. LITTER CONDITION SCORE (for each pen) was determined using the following scale: 0=Litter balls up into a neat ball (without moisture being squeezed out). This is considered ideal litter moisture level; 1=Litter is too dry. Litter DOES NOT ball up into a neat ball; 2=Litter is too wet. Litter balls up, but moisture is squeezed out of the sample.

Bacteria premix stability, following fresh feed mixing bi-weekly, was checked bi-weekly. No significant differences were found in stability.

Significant differences (P>0.05) were observed in weight gain and feed conversion (for both 42 and 84 days of age) when at least 10⁶ Lactobacilli was included in the ration continuously during the growout period. With 10⁷ Lactobacilli, both body weight and feed conversion improved. In general, with increasing levels of Lactobacilli, both weight gain and feed conversion improved. No differences were found in mortality between turkeys in rations containing an increased level of Lactobacilli. Based on the results from this study, it was concluded that the addition of at least 10⁶ Lactobacilli may improve turkey performance as compared to rations without the addition of Lactobacilli. In summary, over the course of 84 days, the supplemented group showed about 3.38% higher weight gain, and 3.76% improved feed efficiency as compared to the control group that received no LAB supplement. Mortality of the subject birds was also reduced from 1.56% in the control group to 1.04% in the supplemented group.

TABLE 22
Body Weight at Day 0

| Criterion | T1 | T2 | T3 | T4 |
|---|---|---|---|---|
| Average Body Wt. (lb) Day 0 | 0.114 | 0.117 | 0.115 | 0.114 |
| Stat¹ | b | a | ab | b |

¹Means within a row without a common superscript are significantly different (P < 0.05) as determined by Least Significant Difference.

TABLE 23
Feed Conversion at Day 0-42

| | Treatment | | | |
|---|---|---|---|---|
| Criterion | T1 | T2 | T3 | T4 |
| Average Body Wt. (lb) Day 42 | 6.729 | 6.733 | 6.826 | 6.974 |
| Stat¹ | b | b | ab | a |
| Feed Conversion Corrected Day 0-42 | 1.452 | 1.436 | 1.425 | 1.382 |
| Stat¹ | c | bc | b | a |
| Mortality % Day 0-42 | 1.04 | 0.52 | 1.04 | 1.04 |
| Stat¹ | a | a | a | a |
| Average Body Wt. Gain (lb) Day 0-42 | 6.615 | 6.616 | 6.712 | 6.860 |
| Stat¹ | c | c | b | a |

¹Means within a row without a common superscript are significantly different (P < 0.05) as determined by Least Significant Difference

TABLE 24
Feed Conversion Day 0-84

| | Treatment | | | |
|---|---|---|---|---|
| Criterion | T1 | T2 | T3 | T4 |
| Average Body Wt. (lb) Day 84 | 22.907 | 22.974 | 23.272 | 23.676 |
| Stat¹ | b | b | ab | a |
| Feed Conversion Corrected Day | 1.856 | 1.851 | 1.833 | 1.794 |
| 0-84 | | | | |
| Stat¹ | c | bc | b | a |
| Mortality % Day 0-84 | 1.56 | 0.52 | 1.04 | 1.04 |
| Stat¹ | a | a | a | a |
| Average Body Wt. Gain (lb) Day | 22.793 | 22.856 | 23.157 | 23.562 |
| 0-84 | | | | |
| Stat¹ | b | b | ab | a |

¹Means within a row without a common superscript are significantly different (P < 0.05) as determined by Least Significant Difference

TABLE 25
Feed Conversion Day 43-84

| | Treatment | | | |
|---|---|---|---|---|
| Criterion | T1 | T2 | T3 | T4 |
| Feed Conversion Corrected Day | 2.027 | 2.026 | 2.005 | 1.970 |
| 43-84 | | | | |
| Stat¹ | b | b | b | a |
| Mortality % Day 43-84 | 0.52 | 0.00 | 0.00 | 0.00 |
| Stat¹ | a | a | a | a |
| Average Body Wt. Gain (lb) Day | 16.178 | 16.240 | 16.445 | 16.702 |
| 43-84 | | | | |
| Stat¹ | b | ab | ab | a |

¹Means within a row without a common superscript are significantly different (P < 0.05) as determined by Least Significant Difference

TABLE 26
Lesion Scores and Litter Condition Scores

| | Treatment | | | |
|---|---|---|---|---|
| Criterion | T1 | T2 | T3 | T4 |
| Average Lesion Score Day 21 | 1.042 | 1.000 | 0.833 | 0.583 |
| Stat¹ | b | b | ab | a |
| Litter Condition Score Day 21 | 0.75 | 0.75 | 0.67 | 0.67 |
| Stat¹ | a | a | a | a |
| Litter Condition Score Day 42 | 1.33 | 1.42 | 1.25 | 0.83 |
| Stat¹ | ab | b | ab | a |
| Litter Condition Score Day 84 | 1.50 | 1.33 | 1.17 | 0.83 |
| Stat¹ | b | ab | a | a |

¹Means within a row without a common superscript are significantly different (P < 0.05) as determined by Least Significant Difference.

Lesion scores on Day 21 were significantly improved with added levels of Lactobacilli over the control treatment. As Lactobacilli levels increased, feed conversion improved and lesion scores decreased. This indicates that improved lesion scores with the use of higher levels of added Lactobacilli may improve turkey performance.

Litter condition, as measured by the categories of Too Dry, Ideal, and Too Wet called “Litter Condition Score”, for birds grown to 84 days were also significantly improved with at least 10⁶ Lactobacilli over the control treatment.

E. coli counts (CFU's, or Colony Forming Units, per ml of carcass rinse solution) and Salmonella incidence (% of turkeys per pen) of processed carcasses were significantly improved with at least 10⁶ Lactobacilli over the control group (Table 27). Salmonella incidence was reduced by 47.8% (P<0.05) for the T4 group (17.92%) as compared to the T1 control group (34.31%). E. coli counts (log 10) were also reduced from 109.05 (T1) to 80.55 (T4).

TABLE 27
Incidence of Pathogen Infections

| | Treatment | | | |
|---|---|---|---|---|
| Criterion | T1 | T2 | T3 | T4 |
| E. coli Count (log10) Day 84 | 109.051 | 102.378 | 90.123 | 80.547 |
| Stat¹ | c | c | b | a |
| Salmonella spp. Incidence (%) | 34.306 | 38.681 | 23.229 | 17.917 |
| Day 84 | | | | |
| Stat¹ | b | b | a | a |

¹Means within a row without a common superscript are significantly different (P < 0.05) as determined by Least Significant Difference.

Each pen was closely monitored, at a minimum of three times per day, to determine overall health, bird behavior and/or evidence of toxicity, and environmental conditions. Temperature (both high and low temperature monitored each time period) was checked at three locations within the growing area employed for this study three times daily. Temperatures observed range from 83-89° F. (Days 0-7), 78-87° F. (Days 8-14), 70-82° F. (Days 15-21), and 66-73° F. (Days 22-84).

Dry Yield (without giblets or WOG) showed significant (P<0.05) improvement over the control when at least 10⁷ Lactobacilli is fed to turkeys. No significant differences were found in Dry Yield between the 10⁵ and 10⁶ Lactobacilli levels.

Total breast yield and major and minor pectoral yield (live weight %) showed significant improvement for birds fed at least 10⁶ Lactobacilli when compared to control. Practically, separation of minor and major pectoral during removal from the body's bone structure is difficult to accomplish, as well as, a smaller amount; consequently, these data should be pooled with major pectoral to define potential statistical differences.

Total breast yield (i.e., combination of both minor and major pectoral) and major pectoral yield (WOG weight %) showed significant improvement for birds fed at least 10⁶ Lactobacilli when compared to control.

TABLE 28
Breast Meat Yield

| | Treatment | | | |
|---|---|---|---|---|
| Criterion | T1 | T2 | T3 | T4 |
| Dry Yield (% WOG) Day 84 | 74.004 | 74.109 | 74.388 | 74.742 |
| Stat¹ | b | b | ab | a |
| Breast Yield (% Live) Day 84 | 16.368 | 16.465 | 16.753 | 17.126 |
| Stat¹ | c | bc | b | a |
| Breast Yield (% WOG) Day 84 | 22.15 | 22.26 | 22.56 | 22.95 |
| Stat¹ | b | b | ab | a |

¹Means within a row without a common superscript are significantly different (P < 0.05) as determined by Least Significant Difference.

TABLE 29
Major and Minor Pectorial meat yield

| | Treatment | | | |
|---|---|---|---|---|
| Criterion | T1 | T2 | T3 | T4 |
| Major Pectorial Yield (% Live) | 12.546 | 12.595 | 12.900 | 13.180 |
| Day 84 | | | | |
| Stat¹ | c | c | b | a |
| Minor Pectorial Yield (% Live) | 3.821 | 3.869 | 3.854 | 3.946 |
| Day 84 | | | | |
| Stat¹ | b | b | ab | a |
| Major Pectorial Yield (% WOG) | 16.98 | 17.03 | 17.37 | 17.66 |
| Day 84 | | | | |
| Stat¹ | b | b | a | a |
| Minor Pectorial Yield (% WOG) | 5.172 | 5.231 | 5.189 | 5.287 |
| Day 84 | | | | |
| Stat¹ | b | ab | ab | a |

¹Means within a row without a common superscript are significantly different (P < 0.05) as determined by Least Significant Difference.

TABLE 30
Breast, Major and Minor Pectorial Meat Yield

| | Treatment | | | |
|---|---|---|---|---|
| Criterion | T1 | T2 | T3 | T4 |
| Breast Yield (Weight per bird, pounds) | 3.796 | 3.836 | 3.944 | 4.102 |
| Stat¹ | d | c | b | a |
| Breast Major Pectorial Yield | 2.910 | 2.934 | 3.037 | 3.157 |
| (Weight per bird, pounds) | | | | |
| Stat¹ | d | c | b | a |
| Breast Minor Pectorial Yield | 0.886 | 0.902 | 0.907 | 0.945 |
| (Weight per bird, pounds) | | | | |
| Stat¹ | d | c | b | a |

¹Means within a row without a common superscript are significantly different (P < 0.05) as determined by Least Significant Difference.

In summary, over the course of 84 days, the supplemented group showed significant improvement in breast meat yield at an average 4.10 pounds for the supplemented group compared to an average 3.80 pounds for the control group that received no LAB supplement.

Considering all data analyzed, Lactobacilli supplements have a significant effect on live performance and other meat yield criteria, especially when placed on built-up litter. Based on all the data generated in this study, the use of at least 10⁶ added Lactobacilli in all rations fed to turkeys is desirable. Lactobacilli bacteria were found to be stable when feeds were mixed bi-weekly. The incidence of E. coli and Salmonella spp. appeared to be reduced with increasing levels of Lactobacilli. E. coli counts (CFU's, or Colony Forming Units, per ml of carcass rinse solution) and Salmonella incidence (% of turkeys per pen) of processed carcasses were significantly improved over the control when at least 10⁶ Lactobacilli were supplemented.

We claim:

1. A method for improving feed utilization or reducing pathogen in a bird, said method comprising:
(a) administering to said bird a composition comprising a lactic acid producing bacterium, said lactic acid producing bacterium being supplemented to the bird at a dosage range of between 1×10³ and 1×10¹⁰ CFU of lactic acid producing bacterium per day for each bird, wherein the lactic acid producing bacterium is a Lactobacillus strain selected from the group consisting of LA51, M35, LA45, NP28, and L411 strains.

2. The method of claim 1, wherein the dosage is from about 1×10⁶ to about 1×10⁹ CFU of lactic acid producing bacterium per day for each bird.

3. The method of claim 1, wherein the dosage is from about 1×10⁷ to about 1×10⁸ CFU of lactic acid producing bacterium per day for each bird.

4. The method of claim 1, wherein said bird is selected from the group consisting of a chicken, a laying hen, a duck, a goose, a turkey, a fowl and a pheasant.

5. The method of claim 1, wherein said bird is a domesticated bird and is in need of said supplement of lactic acid producing bacterium.

6. The method of claim 1, further comprising a step (b) of measuring or predicting performance of said bird to determine if said bird is in need of said supplement of lactic acid producing bacterium, said step (b) preceding said step (a).

7. The method of claim 1, further comprising a step (c) of measuring performance of said bird to assess the effect of said supplement of lactic acid producing bacterium, said step (a) preceding said step (c).

8. The method of claim 6, further comprising a step (d) of measuring the amount of a pathogen in said bird to assess the effect of pathogen reduction by said supplement of lactic acid producing bacterium, said step (a) preceding said step (d).

9. The method of claim 1, wherein said bird is a broiler, and said step (a) is performed for a period of between 20 and 50 days.

10. The method of claim 1, wherein said bird is a turkey, and said step (a) is performed for a period of about 80-120 days.

11. The method of claim 1, wherein said bird is a hen, and said step (a) is performed for at least 300 days.

12. The method of claim 1, wherein said step (a) is performed continuously on a daily basis.

13. The method of claim 7, wherein said step (c) is performed at least 2 weeks after said step (a), and wherein feed efficiency of said bird is measured in step (b) and step (c), and the feed efficiency obtained in step (c) is at least 2% better than that obtained in said step (b).

14. The method of claim 1, wherein the feed efficiency of said bird is at least 2% better than the feed efficiency of an unsupplemented bird.

15. The method of claim 14, wherein the feed efficiency of said bird is at least 3% better than the feed efficiency of an unsupplemented bird.

16. The method of claim 7, wherein breast meat content of said bird is measured in step (b) and step (c) and said breast meat content in step (c) is at least 1% higher than that obtain in step (b).

17. The method of claim 1, wherein breast meat content of said bird is at least 1% higher than that of an unsupplemented bird.

18. The method of claim 17, wherein breast meat content of said bird is at least 3% higher than that of an unsupplemented bird.

19. The method of claim 17, wherein breast meat content of said bird is at least 6% higher than that of an unsupplemented bird.

20. The method of claim 1, wherein said composition being supplemented to the bird does not contain significant amount of lactic acid utilizing bacterium.

21. The method of claim 1, wherein said bird is raised on built-up litter.

22. The method of claim 1, wherein said lactic acid producing bacterium at said dosage reduces infection of said bird by a pathogen by at least 20% when compared with infection by the same pathogen in an unsupplemented bird.

23. The method of claim 22, wherein said pathogen is selected from the group consisting of Salmonella typhimurium, E. coli, Staphylococcus aureus and Campylobacter jejuni.

24. The method of claim 23, wherein said pathogen is E. coli O157:H7.

25. (canceled)

26. The method of claim 1, wherein the Lactobacillus strain is the LA51 strain.

27. The method of claim 1, wherein said bird is a laying hen and said lactic acid producing bacterium is supplemented to the hen at a dosage sufficient to reduce the amount of at least one pathogen on the exterior surface of eggs produced by the hen by at least 30% as compared to the amount of said at least one pathogen on the exterior surface of eggs produced by an unsupplemented bird.

28. The method of claim 1, wherein said bird is a laying hen and said lactic acid producing bacterium is supplemented to the hen at a dosage sufficient to reduce the amount of at least one pathogen on the exterior surface of eggs produced by the hen by at least 60% as compared to the amount of said at least one pathogen on the exterior surface of eggs produced by an unsupplemented bird.

29. The method of claim 1, wherein said bird is a laying hen and said lactic acid producing bacterium is supplemented to the hen at a dosage sufficient to reduce the amount of at least one pathogen in the oviduct of the hen by at least 30% as compared to the amount of said at least one pathogen in the oviduct of an unsupplemented bird.

30. The method of claim 1, wherein no antibiotic is administered to the bird.

31. The method of claim 1, wherein said bird is a laying hen and the lactic acid producing bacterium is supplemented to the bird at a dosage of from 1×10⁶ CFU to 1×10⁷ CFU per day for each bird.

32. The method of claim 31, wherein the lactic acid producing bacterium is supplemented to the bird at a dosage of about 1×10⁷ CFU per day for each bird.

33. A composition for improving feed utilization by a bird, said composition comprising a lactic acid producing bacterium, said lactic acid producing bacterium being supplemented to said bird at a dosage of between 1×10³ and 1×10¹⁰ CFU per day for each bird, wherein said lactic acid producing bacterium is a Lactobacillus strain selected from the group consisting of LA51, M35, LA45, NP28, and L411 strains.

34. (canceled)

35. The composition of claim 33, wherein the Lactobacillus strain is the LA51 strain.

36. The composition of claim 33, wherein said bird is a domesticated bird.

37. The composition of claim 33, wherein said lactic acid producing bacterium is pre-mixed with feed or water for said bird.

38. The composition of claim 36, wherein said lactic acid producing bacterium at said dosage improves feed efficiency of said domesticated bird by at least 2%.

39. The composition of claim 36, wherein said lactic acid producing bacterium at said dosage improves feed efficiency by said domesticated bird by at least 3%.

40. The composition of claim 36, wherein said lactic acid producing bacterium at said dosage improves feed efficiency by said domesticated bird by at least 4%.

41. The composition of claim 36, wherein said lactic acid producing bacterium at said dosage reduces infection of said domesticated bird by a pathogen.

42. The composition of claim 41, wherein said pathogen is selected from the group consisting of Salmonella typhimurium, E. coli, Staphylococcus aureus and Campylobacter jejuni.

43. The composition of claim 33, wherein said composition does not contain significant amount of lactic acid utilizing bacterium.

44. A method for improving feed utilization or reducing pathogen in a bird, said method comprising administering to said bird a composition comprising a Lactobacillus strain LA51, said Lactobacillus strain being supplemented to the bird at a dosage of from about 1×10³ to about 1×10¹⁰ CFU per day for each bird.
